(19) United States
(12) Patent Application Publication
(10) Pub. No.: US 2003/0162207 A1
(43) Pub. Date: Aug. 28, 2003

(54) Multi-gene tests with ROC plots for the assessment of risk for polygenic disorders

(76) Inventors: David E. Comings, Duarte, CA (US); James P. MacMurray, Loma Linda, CA (US)

Correspondence Address: ROTHWELL, FIGG, ERNST & MANBECK, P.C., 1425 K STREET, N.W., SUITE 800, WASHINGTON, DC, 20005, US

(21) Appl. No.: 10/319,855

(22) Filed: Dec. 16, 2002

Related U.S. Application Data
(60) Provisional application No. 60/339,426, filed on Dec. 14, 2001.
(60) Provisional application No. 60/413,775, filed on Sep. 27, 2002.

(51) Int. Cl. C12Q 1/68

(52) U.S. Cl. 435/6

(57) ABSTRACT

Polygenic disorders are due to the additive effect of multiple genes interacting with the environment. Because of the small effect size of each gene and considerable genetic heterogeneity, when single genes are examined, the outcome of association and linkage analyses are variable from study to study. Techniques are needed that take these unique characteristics of polygenic disorders into consideration. The present invention discloses that the formation of a polygenic score, consisting of the additive effect of multiple candidate genes, and its assessment using receiver operating characteristic (ROC) plots, provides such a technique. Six genes previously shown to be associated with Alzheimer's disease were examined, APOE, ACE, ACP1, ESR1, PNMT and SLC6A4. The total fraction of the variance, the area under the ROC plots, and the range of risks were similar for both groups indicating that despite genetic heterogeneity and the small effect size of most genes, consistent risk analyses could be obtained by examining the additive effect of these multiple genes. The present invention also discloses diagnostic tests for determining a subject's risk of developing Alzheimer's Disease or specifically Late Onset Alzheimer's Disease.

### CROSS-REFERENCE TO RELATED APPLICATIONS

The present application is related to and claims priority under 35 U.S.C. 119(e) to U.S. provisional patent application Serial No. 60/339,426 filed Dec. 14, 2001 and Serial No. 60/413,775 filed Sep. 27, 2002.

### FIELD OF INVENTION

This invention relates generally to the field of human genetics and methods of detecting multiple genes that contribute to polygenic traits, diseases or disorders. More specifically, the invention relates to polygenic assays for detecting an increased risk in a subject to developing Alzheimer's disease.

The publications and other materials used herein to illuminate the background of the invention or provide additional details respecting the practice are incorporated by reference and for ease of reference are included in the Bibliography.

### BACKGROUND AND SIGNIFICANCE

Most of the disorders that affect humans are polygenically inherited. Polygenic disorders are due to the additive and epistatic effect of multiple genes (polygenes) (Comings 1998), each with a small effect, interacting with the environment. By contrast, in single gene disorders each mutation accounts for essentially 100 percent of the variance. However, single gene disorders collectively account for less than 2 percent of all disease morbidity, while virtually all humans are at risk during their lifetime for one or more polygenic disorders. While lod score and sibling pair linkage studies have been very successful in identifying the loci involved in single gene disorders, they lack the power to identify most polygenes (Risch 2000). Family based or population based association studies do have the power to identify genes with a small effect size. However, poor replication between studies is the rule in polygenic disorders (Ioannidis et al. 2001; Hirschhorn et al. 2002). This has often been blamed on population stratification (Hamer and Sirota 2000). Family based studies such as transmission/disequilibrium test (TDT) (Spielman and Ewens 1996) have been proposed to eliminate this problem. However, as the number of association studies has grown, it has become clear that the variability from study to study is just as great with family based as with population based studies, suggesting that population stratification was not the culprit. Since the TDT technique requires samples on both parents and an affected child and is most powerful where there is one heterozygous and one homozygous parent, independent of its ability to negate possible population stratification, it is less powerful than case control association studies (Morton and Collins 1998). In addition, because of the need for a heterozygote parent for each gene, if the additive effect of multiple genes is to be examined, the TDT technique is dramatically weakened compared to case controls studies.

Case control studies are also especially valuable in cases where older individuals are affected, such as Alzheimer's disease or cancer, or behavioral disorders, where as a result of family conflicts, obtaining both parents may be difficult. Thus, for many, and possibly all polygenic disorders, especially if the additive effect of multiple genes is to be examined, case control studies are the most powerful approach to the identification of the causative genes. However, concerns about the potential for population stratification remain. To counter this, several authors have proposed genotyping a number of non-candidate genes to identify and correct for possible population stratification (Devlin and Roeder 1999; Devlin, Roeder, and Bacanu 2001; Zhang and Zhai 2001; Reich D E and Goldstein D B 2001).

In previous studies of several hundred genotype-phenotype associations, even when significant, the percent of the variance attributable to each gene generally ranged from 0.5 to 3.0 percent and averaged less than 1.5 percent (Comings et al. 2000a,b,c; 2001). Even in disorders that are only 50 to 70 percent genetic, this implies the potential involvement of many polygenes. A second major characteristic of polygenic disorders is great genetic heterogeneity. Thus, the same phenotype may be caused by many different combinations of polygenes. It has proposed that the interaction of these two characteristics, a small percent of the variance attributable to each gene and great genetic heterogeneity, is the true cause of the variation from study to study and is the expected outcome for polygenic disorders (Comings 2002). Thus, instead of focusing on single genes and imperfect replication from study to study, this phenomenon should be recognized as an integral and expected outcome for polygenic disorders and objective should instead focus on developing methods that take the unique characteristics of single genes into consideration. One of the most challenging aspects of polygenic disorders is that they are due to the additive effect of multiple genes each of which has only a small effect, and there is considerable heterogeneity such that the involved genes may differ from one study group to another. Different sets of genes may produce the same phenotype. As a result, when genes are examined one-at-a-time the results are often significant in some studies but not in others, i.e., replication is often poor.

One potential approach to examining polygenic disorders is to examine the total variance of functionally related genes. A number of different but functionally related genes can each contribute to the same phenotype. For example, several different dopamine genes, DRD1 (Comings et al. 1997), DRD2 (Comings et al. 1991), DRD3 (Comings et al. 1999) DRD4 (Lahoste et al. 1996; Rowe et al. 1998; Faraone et al. 1999), DRD5 (Daly et al. 1999), SLC6A3, DAT1 (Ebstein et al. 1996; Comings et al. 1996; Gill et al. 1997; Waldman et al. 1998), DOC (Ernst et al. 1998), SLC18A1, and SLC18A2 (Russlee et al. 1998) have all been implicated in attention deficit hyperactivity disorder or its comorbid conditions, such as substance abuse. However, replication from study to study has been far from perfect. This suggests that a more reasonable approach is to examine the variance of each dopamine gene and compare the sum of the variance for all dopamine genes in subjects with the phenotype versus controls. The potential epistatic interaction between the dopamine genes could also be included in the total variance. To further characterize a given disorder, the relative total variance of genes belonging to different functional groups of genes, such as dopamine, serotonin, norepinephrine, GABA, opioid, cholinergic, etc., can be examined for different phenotypes. This approach has been used in the examination of the role of multiple genes in ADHD, oppositional defiant disorder (ODD), conduct disorder (CD), pathological gambling, and personality traits (Comings et al. 2000a,b,c; 2001).

Another potential approach to determine contribution to polygenic disorders is to determine multigene additive scores by ROC plots. The analysis of total variance approach may require the examination of hundreds of genes. While this is increasingly practical with high throughput genotyping techniques, some disorders may be more efficiently studied by examining the additive and epistatic effect of a small number of functionally different candidate genes. It is often the case that a number of genes may have already been implicated in a given disorder and while some show good or excellent replication across multiple studies, the results for others may show a mixture of replication and non-replication.

Alzheimer's disease is the most common cause of dementia and affects roughly four million individuals. The ultimate goal of understanding the causes of Alzheimer's disease is to identify better methods of treatment and prevention. About 5 to 12% of Alzheimer's disease is due to autosomal dominant genes. The genes involved include the AD1 locus for the APP gene on chromosome 21 (St George-Hyslop et al. 1987) that codes for the Amyloid Precursor Protein (APP) (Karlinsky et al. 1992) and contains the sequences of amyloid-(A) present in senile plaques, the AD3 locus for PS-1 gene on chromosome 14 (Schellenberg et al. 1992; Alzheimer's Disease Collaborative Group 1995; Sherrington et al.1995;Sandbrink et al.1996) and the AD4 locus for PS-2 on chromosome 1 (Levy-Lahad et al.1995; Rogaev et al.1995). The majority of Alzheimer's disease cases are sporadic. The AD2 locus for the APOE gene on chromosome 19 is an important locus for sporadic Alzheimer's disease (Saunders et al.1993; Saunders et al. 1996). The three major alleles are e2, e3 and e4, differing in two residues at position 112 and 158. The e4 allele is associated with an increased risk for Alzheimer's disease. It is present in 14% of normal Caucasians versus 37% in sporadic late-onset Alzheimer's disease (LOAD), and 48% in LOAD subjects with a family history of Alzheimer's disease (Saunders et al. 1993; Poirier et al.1993; Strittmatter et al. 1993). PS-1 has also been suggested as a risk factor for some cases of LOAD.

Based on studies of a number of different countries, factors such as dietary fat and total caloric intake have been shown to be highly correlated with the prevalence of Alzheimer's disease (Grant 1997). While this might seem to imply that environmental factors are more important than genetic ones, it is more likely that this represents the results of genetic environmental interaction. For example, a high fat diet may produce oxidative stress, and individuals genetically susceptible to such stress may be the ones who develop Alzheimer's disease. In the absence of a high fat diet the oxidative stress is too small to produce Alzheimer's disease even in genetically susceptible individuals.

In the present invention the risk of having Alzheimer's disease (AD) or in particular Late Onset Alzheimer's Disease (LOAD) is determined. APOE is the prime example of a risk gene for a sporadic, polygenic disorder that has shown a high degree of replication across many studies (Farrer et al. 1997). In addition, there have been a number of other genes that have shown a significant association with AD in some, but not all studies. Despite the demonstration of strong association of the APOE gene with LOAD, it still has not been recommended as a screening test for AD (ACMG/ASHG 1995).

### SUMMARY OF THE INVENTION

The present invention relates the detection of a set of genes that together cause a polygenic disorder. Polygenic disorders are due to the additive effect of multiple genes, each of which has only a small effect, and there is considerable heterogeneity such that the involved genes may differ from one study group to another. Different sets of genes may produce the same phenotype.

In one embodiment, the invention provides a method for determining whether a group of genes together contribute to a polygenic trait, disease or disorder, the method comprising the steps of: (1) genotyping two or more candidate genes having an allele significantly associated with the polygenic trait, disease or disorder; (2) scoring one or more alleles for each gene depending upon whether the allele for the gene showed the least, intermediate, or strongest association with the polygenic trait, disease or disorder; (3) performing multivariate logistic regression analysis to determine which of the alleles of said genes, in the presence of the other alleles of said other genes, continued to contribute to the polygenic trait, disease or disorder; (4) determining a combined relative risk score based on adding together the genes selected by the logistic regression analysis; and (5) examining the combined relative risk score in a Receiver Operator Characteristic (ROC) plot, wherein the ROC curves plot the different values of the test against the specificity and 1—the sensitivity of each value and wherein if a combined relative risk score is greater than the risk score associated with an individual allele for two or more of said genes, the detection of said two or more genes represent an improved test for predicting the presence or predisposition to said trait, disease or disorder when compared to a method which detects only one of said alleles of said genes. In another embodiment, the process further comprises the step of (6) repeating the entire process in a second, well-matched set of subjects having the polygenic trait, disease or disorder and controls to determine the degree to which the results in the first set could be replicated in the second set. Once a set of genes found to be associated with a polygenic trait, disease or disorder is determined, an assay for determining whether an individual is at an increased risk for developing or having the trait, disease or disorder can be developed, wherein said assay comprises analyzing an individual's genetic material for the presence of said two or more alleles from said two or more genes.

The present invention provides AD-risk associated and LOAD-risk associated genes which, even though they do not account for as much of the variance of LOAD as APOE, when used in an additive fashion with or without the APOE gene, sufficiently increase the predictability of AD or LOAD to be clinically useful. Thus, in another embodiment, the invention provides methods or determining whether an individual is at an increased risk of developing Alzheimer's disease, the method comprising analyzing an individual's genetic material for the presence of two or more AD-risk associated or LOAD risk associated alleles.

In one embodiment, the invention provides a kit suitable for screening a subject to determine whether such subject is at increased risk for having or developing AD associated with the presence of a AD-risk associated gene, said kit comprising material for determining the subject's genotype with respect to at least two AD-risk associated genes; suitable packaging material; and optionally instructional material for use of said kit.

In another embodiment, the invention provides a method for developing a polygenic assay that is diagnostic for a trait, disease or disorder which comprises identifying the trait, disease or disorder that is to be studied; creating a scale measuring the lowest and highest scores representing the phenotypic expression of the trait, disease or disorder to be studied; selecting at least two candidate genes that may contribute to said trait, disease or disorder; identifying at least one polymorphic allele associated with each of said candidate genes and the trait, disease or disorder; correlating allelic patterns of said polymorphism with said scale; comparing the association of said allelic pattern to the correlation of said candidate gene to said trait, disease or disorder; wherein the allelic patterns that are positively associated with said trait, disease or disorder are added, to form a polygenic assay that is diagnostic for the trait, disease or disorder; wherein the assay comprises detecting the presence of the allelic patterns that are positively associated with said trait, disease or disorder.

### BRIEF DESCRIPTION OF THE FIGURES

FIG. 1A shows a graphical representation of the ROC curve for the APOE gene and LOAD.

FIG. 1B shows a graphical representation of the ROC curve for all genes examined excluding the APOE gene for LOAD.

FIG. 1C shows a graphical representation of the ROC curve for all genes examined for LOAD.

FIG. 1D shows a graphical representation of the ROC curve for the weighted relative risk scores for all genes examined.

FIG. 2 shows a scatterplot analysis of the relative risk scores for LOAD cases and controls.

FIG. 3 shows a graphical representation of the ROC curve for relative risk scores of APOE associated with AD.

FIG. 4 shows a graphical representation of the ROC curve for relative risk scores for all genes examined for AD.

### DETAILED DESCRIPTION OF THE INVENTION

The first step in developing a multi-gene test for risk of a disease is to genotype candidate genes that are significantly associated with the disease or disorder. In exemplary embodiments, the present invention presents such methods wherein the disease or disorder is AD or LOAD. In these examples, candidate genes that each individually had been significantly associated with AD or LOAD were chosen for further analysis. In analyzing genes suggestive of an association with AD or LOAD, all genes were scored 0 to 2 depending upon whether a given genotype showed the least, intermediate, or strongest association with AD or LOAD. The next step was to utilize multivariate logistic regression analysis to determine which of the genes, in the presence of the other genes, continued to contribute to AD or LOAD. Next, a combined relative risk score for all of the genes discovered was determined based on adding together the scores of all of the genes selected by the logistic regression analysis. The combined relative risk score was then analyzed in a Receiver Operator Characteristic (ROC) plot. A critical aspect of any test is to determine both its specificity and sensitivity. ROC curves plot the different values of the test against the specificity and 1-sensitivity of each value. The entire process was repeated in a second, well-matched set of LOAD subjects and controls to determine the degree to which the results in the first set could be replicated in the second set.

When performing the above delineated process, candidate genes with a strong and consistently replicated association with e.g., LOAD, such as the APOE gene, should show a significant association with LOAD in both sets. The remaining genes, known to be significantly associated with LOAD in at least one study, but with a more modest association with LOAD, should fall into one of three groups: 1) significant association with LOAD for both groups; 2) significant for one group but not the other; and 3) significant for neither group. Despite the set-to-set variability of the individual genes, due to the ability of one risk gene to substitute for another risk gene, the total variance for both sets would be comparable. Adding the gene scores and examining the resulting combined relative risk score using ROC plots would produce reasonably similar results for both sets. Despite the two difficult characteristics of polygenic disorders, of a small effect size of each gene and considerable genetic heterogeneity, examining multiple genes and combining their effect into a single risk score would help to circumvent these characteristics and provide a valuable method of examining an individual's risk for a given complex disorder.

General Methods

AD Cases. To test the validity of the use of a number of different genes as predictors of the presence or absence of AD we have utilized two resources, a sample of autopsy proven cases of AD, and a sample of age and race matched controls.

AD Sample. This sample consists of 154 cases of autopsy proven AD cases. If a cutoff of 64 years of age is used, they consist of 46 early onset AD cases and 108 late onset cases. If a cutoff of 69 years of age is used, they consist of 70 early onset and 83 late onset cases. For the whole group the age at death ranged from 55 to 97 years with a mean of 76.1 S.D 9.2 years, while the age at onset ranged from 49 to 92 years with a mean of 70.2 S.D. 9.8 years. The samples were obtained from the Human Neurological Research Specimen Bank at Los Angeles Veterans Affairs Medical Center, Los Angeles, Calif. All cases have been approved for use in the bank by IRB approved protocol and consent forms signed by relatives. In each case the diagnosis was verified by histopathological examination. The study was limited to Caucasians. DNA was isolated from the brain samples using standard techniques.

AD Controls. This sample consisted of 283 Caucasians 56 years of age or greater. They were derived from individuals with non-dementing conditions and without psychiatric disorders as assessed by a number of standardized instruments. All studies were approved by IRB committees and each subject signed a consent form. DNA was obtained from blood leukocytes from a sample of 7 to 14 ml of blood anticoagulated with EDT A. The age of the controls ranged from 56 to 96 with a mean of 65.5 S.D. 7.76 years.

LOAD Cases. The LOAD case in this study is defined as age of onset of AD greater than 65 years. All of the cases are from Alzheimer's disease brain banks with a histologically proven diagnosis of AD. There were a total of 204 LOAD cases. The brain banks and the total number of samples from each, were the following: (1) 160 were from the Human Neurological Research Specimen Bank at Los Angeles Veterans Affairs Medical Center, Los Angeles, Calif.; (2) 96 cases were from the Rush Medical Center Alzheimer's Disease Brain Bank, Chicago, Ill., Dr. David A. Bennett (these included samples from the Catholic nuns study, the Rush Medical Center clinical core and community referrals); and (3) 40 cases were from the LSU Neuroscience Center Brain Tissue Bank, New Orleans, La., Dr. Hector LeBlanc. From these we selected all cases that could be definitively diagnosed as LOAD cases.

DNA isolation from brain samples. One gram of brain tissue was homogenized in cold 0.075 M NaCl, 0.25 M EDTA (pH 7.0). After centrifugation at 1000×g, the pellet was washed twice with cold 0.1 M Tris and 0.1 M EDTA (TE) (pH 8.0). The pellet was re-suspended in 5 ml of 0.01 M TE and 0.05 M SDS and incubated at 56° C. with 200 ul of protease K (10 mg/ml) for 48 hours. To stop the reaction, 5 ml of 7.5 M ammonium acetate was added to the solution and mixed well. The DNA was precipitated by adding two volumes of 70% isopropanol and collecting by centrifugation. The pellet was washed with 100% ethanol and collected by centrifugation. The vacuum-dried pellet was re-suspended in TE buffer by shaking overnight. The DNA was quantified spectrophotometrically.

LOAD Control Subjects. DNA was obtained from blood leukocytes from 298 control samples which consisted of the following four cohorts: (1) 29 controls were from the Human Neurological Research Specimen Bank at Los Angeles Veterans Affairs Medical Center, Los Angeles, Calif. (these were subjects dying from accidents, or non-dementing medical conditions and histologically proven not to have AD); (2) 119 adult out-patients from Loma Linda University Center for Health Promotion (these were healthy individuals attending the center for general health promotion issues); (3) 47 adult in-patients from the Jerry L. Pettis Veterans hospital (these individuals had a range of non-dementing, non-neurological medical conditions; (4) 89 veterans over 55 years of age who had volunteered for a national athletic competition called the Golden Age Games; and (5) 14 adopting parents of children from the Tourette syndrome clinic. All controls were non-Hispanic Caucasians with no history of AD or other dementing disorders.

Replication. LOAD subjects and controls randomly into two sets with 250 individuals in each set. To form two groups matched by diagnosis, sex and age, all cases in a spreadsheet were sorted by diagnosis, then sex, then age, and alternate cases assigned to set 1 and set 2.

All studies were approved by the IRB committees of the involved institutions and each subject or, in the case of the brain samples each subjects relatives, signed a consent form.

Genes and Alleles Studied. APOE. The APOE 2/3/4 polymorphism has consistently been used as an example of an association with LOAD study that has been replicated in virtually all studies (Farrer et al. 1997). Apolipoprotein E, a main apoprotein of the chylomicron, binds to a specific receptor on liver cells and peripheral cells. It is essential for the normal catabolism of triglyceride-rich lipoprotein constituents. The APOE gene is mapped to chromosome 19 in a cluster with APOCI and APOC2. Defects in apolipoprotein E result in familial dysbetalipoproteinemia, or type III hyperlipoproteinemia (HLP III), in which increased plasma cholesterol and triglycerides are the consequence of impaired clearance of chylomicron and VLDL remnants. Apolipoprotein E (APOE) is a major apoprotein of the chylomicrons. It binds to a specific receptor on liver cells and peripheral cells and is essential for the normal catabolism of triglyceride-rich lipoprotein constituents. It is involved in the mobilization and redistribution of cholesterol in repair growth, and maintenance of myelin and neuronal membranes during development and after injury 8. APOE is present in the senile plaques of AD 9. The association of the epsilon 2,3, and 4 alleles with AD has been replicated in many different studies. A recent meta-analysis of results from 40 centers, involving 5,930 subjects, indicated that the AOPE e4 allele represents a major. risk factor for AD in all ethnic groups studied across ages between 40 and 90 years, in both men and women 10. Among Caucasians the average odds ratios were e2/e4 (OR=2.6, 95% CI=1.6-4.0), e3/e4 (OR=3.2,95% CL=2.8-3.8), e4/e4 (OR=14.9,95% CI 10.8-20.6). Despite the universal agreement that the APOE gene is a significant risk gene for AD, there is also consensus that the use of APOE genotyping alone is not suitable for genetic screening for AD risk II. The is in part due to the fact that despite the very high and significant odds ratio for e4/e4 carriers, these individuals are rare in the general population, occurring in only 193 of 4858 or 3.9% of Caucasians (Farrer et al., 1997). While the frequency of e3/e4 carriers was higher (25%) was higher, the odds ratio was lower. The APOE genotyping was performed by the technique of Hixson and Vernier using the restriction endonuclease Hha I (Hixson and Vernier 1990; Appel, Eisenberg, and Roitelman 1995).

Serotonin. A role of defects in serotonin have been implicated in AD 12. Most of the studies of the potential role of genetic variations of serotonin genes in AD have centered on the serotonin transporter gene (SLC6A4), and of these virtually all examined the promoter 5-HTTPR insertion/deletion polymorphism 13-18. Some were positive and some were negative. The majority of the positive studies showed an association with the deletion (S) allele which in turn is associated with decreased expression of the scrotonin transporter. In some reports the association was more with comorbid behaviors such as aggression or psychosis than with AD per se. The VNTR polymorphism in the second intron of the SLC6A4 gene was genotyped by the technique of Battersby et al (1996). The STin2.10 and STin2.12 were the most common alleles. SLC6A4. The promoter insertion/deletion polymorphism of the serotonin transporter gene (5-HTTPLR) has been reported to be associated with AD in some (Oliveira et al. 1998; Li et al. 1997; Mossner et al. 2000) but not all (Kunugi et al. 2000; Zill et al. 2000; Tsai et al. 2001; Oliveira et al. 1999) studies. We have utilized a second major polymorphism of the SLC6A4, a 17 bp VNTR in the second intron (Ogilvie et al. 1996). The major alleles 9, 10 and 12 named for the number of repeats. The 12/12 genotype as been reported to be associated with a lower affinity for platelet serotonin uptake (Kaiser et al. 2002). In preliminary studies we observed a significant association between LOAD and the SLC6A4 gene using the VNTR polymorphism. Some studies of the role of serotonin in AD were positive and some were negative. The majority of the positive studies showed an association with the deletion (S) allele which in turn is associated with decreased expression of the serotonin transporter. In some reports the association was more with comorbid behaviors such as aggression or psychosis than with AD per se. We have chosen to examine the VNTR polymorphism of (SEQ ID NO:1) 5′-GGCTGYGACCY[R]GRRTG-3′ in the second intron of the SLC6A4 gene. This polymorphism has been reported to be associated with depression but has not been previously studied in AD. The 5′ primer is (SEQ ID NO:2) 5′-GTCAGTATCACAGGCTGCGAG-3′, the 3′ primer is (SEQ ID NO:3) 5′-TGTTCCTAGTCTTACGCCAGTG-3′. In the present study the genotypes were 12/12, 12/non12 and non12/nonI2.

ACP1. Acid phosphatase (ACP1) is a ubiquitous enzyme present in all tissues including the brain (Tanino et al. 1999). ACP1 is also known as low molecular weight protein tyrosine phosphatase (LMWPTP). Biochemical analysis and studies with specific antibodies to LMWPTP have shown that the level of ACP1 protein is significantly decreased in AD brains (Shimohama et al. 1995; Shimohama et al. 1993). Genetic variants of ACP1 have been recognized for many years (Spencer, Hopkinson, and Harris 1964). ACP1*A differs from ACP1*B and *C by the presence of an Arg 105 Gln substitution (Dissing and Johnsen 1992). A T->A polymorphism of ACP1*A has been identified creating a Taq I restriction endonuclease site that allows PCR based genotyping of ACP1 (Sensabaugh and Lazaruk 1993). Since ACP1*A has a lower enzyme activity than ACP1*B or *C (Spencer, Hopkinson, and Harris 1964), there is a progressive decrease in ACP1 enzyme activity progressing from Taq I genotypes 11 (absence of *A variant) to 12 (50% *A variant) to 22 (100% *A variant). Genotyping of the ACP1*A Taq I (A>216G; CAA>CGA; Gln>105Arg) polymorphism was performed utilizing the primers set forth in (SEQ ID NO: 4) 5′-TTCAGAAGACCCTAGCAGATG-3′ and (SEQ ID NO:5) 5′-ACATAATAGGGATCTTCGATAATAAG-3′ (Sensabaugh and Lazaruk 1993). The accession number of the ACP1*A gene is L06508. Following amplification, samples were digested with TaqI restriction enzyme and the digested samples were analyzed by electrophoresis through 10% acrylamide gels at 180 volts. Using these methods, the ACP1*A allele generates a 110 base pair fragment while a non-ACP1*A allele generates a 190 base pair fragment. The TaqI restriction enzyme site characteristic of the ACP1*A allele has been described previously and is characterized as a CAA>CGA substitution at codon 105, which creates a Gln>Arg substitution at this codon (Sensabaugh et al. 1993)). Using the Taq I polymorphism, we have reported associations between the ACP1 gene and the metabolic syndrome (Bottini et al. 2002a), teen age smoking (Comings et al. 2002a), depression (Comings et al. 2002b), Tourette syndrome (Bottini et al, 2002) and multiple sclerosis (Bottini et al. 2002b). This association with a range of metabolic and neuropsychiatric disorders and a positive preliminary study for early onset AD (EOAD), led us to include it in the present study.

The angiotensinogen converting enzyme (peptidyl-dipeptidase A) is responsible for the conversion of angiogensinogen I to angiogensinogen II and degrades bradykinin. An insertion-deletion (IID) polymorphism at the ACE gene has frequently been studied as a risk factor in coronary artery disease, hypertension, vascular and Alzheimer's dementia. The are both positive 37.38 and negative studies 39, and both the deletion (D) allele 37,38 and the insertion (I) allele 40-42 have been associated with AD. A negative heterosis effect consisting of a greater association with the I/I and DID homozygotes than IID heterozygotes has also been reported 43. A meta-analysis of 12 studies suggested a 1/1>IID>DID relationship 43. Instead of using the IID polymorphism we have utilized our own polymorphism, a “C>T” variation at position 22251 of Accession number AFI18569. By immunostaining, the levels of ACE (angiotensin-converting enzyme, dipeptidyl carboxypeptidase 1) were found to be elevated in the cortical level V area of individuals with AD (Savaskan et al. 2001). The potential association of the intron 16 insertion/deletion polymorphism (I/D) of ACE gene with Alzheimer's disease has been examined in a number of studies with both positive and negative results (Narain et al. 2000; Isbir et al. 2001; Zuliani et al. 2001; Farrer et al. 2000; Hu et al. 1999; Alvarez et al. 1999). A meta-analysis of 12 studies was consistent with an association with the I allele (p=0.00002) (Narain et al. 2000). In our own pilot studies we found a significant association between LOAD and a C/T 22251 polymorphism of ACE which is in partial but not complete linkage disequilibrium with the I/D polymorphism (Rieder et al. 1999). We utilized detection of the C/T 22251 polymorphism (Accession number AF118569) of the DCP1 gene. The genotyping was performed using a 19 bp forward primer of (SEQ ID NO:6) 5′-GCATCTACACAGG/CCACGGC-3′ while the 21 bp reverse primer was (SEQ ID NO:7) 5′-GAACTGGATGATGAAGCTGTC-3′. The PCR reaction was performed through 35 cycles by the following steps: denaturation at 94° C. for one minute, annealing at 55° C. for one minute, and extension at 72° C. for one minute. The PCR reaction was followed by Taq I endonuclease cleavage. The amplified DNA sequence and the size of the restriction enzyme digested produce were 260 bp and 240 bp for 11 and 22 genotypes respectively. The restriction endonuclease used was Taq I.

Estrogen receptor gene. Epidemiological studies have shown that women who have taken estrogen for a number of years are at significantly decreased risk for AD. This has stimulated interest in the potential role of genetic variants at the estrogen receptor genes as a risk factor for AD 20,21. Several studies have demonstrated an association with the ER alpha (ESR1) and ER beta (ESR2) genes independently and together 22-24. Some studies were negative 25. We examined the Xba I polymorphism of the ER alpha gene 26. The 5′ primer was (SEQ ID NO:8) 5′-TCAGAACCATTAGAGACCAATGC-3′ and the 3′ primer was (SEQ ID NO:9) 5′-AGGGTTATGTGGCAATGACG-3′. This produced a 178 bp PCR product. A number of studies have implicated the taking of estrogen in women as a preventive factor in AD (Tang et al. 1996; Paganini-Hill and Henderson 1996). This has led to studies of the possible association of estrogen receptor 1 (alpha) gene with AD, several of which were positive (Ji et al. 2000; Mattila et al. 2000; Lambert et al. 2001; Brandi et al. 1999), and some of which were negative (Maruyama et al. 2000). The Pvu II and Xba I polymorphisms were most often studied. In the present study we have used both of these polymorphism of the ESR1 gene The Pvu II and Xba I polymorphisms of the ESR1 gene were performed by the technique of Kobayashi et al (Kobayashi et al. 1996).

PNMT. Phenylethanolamine N-methyltransferase is the rate-limiting enzyme in the synthesis of epinephrine from norepinephrine (Axelrod and Weinshilboum 1972). Burke and colleagues (Burke et al. 1987) have shown that PNMT levels are decreased in the brains of persons with AD when compared to normal healthy control brains. We previously identified two promoter region polymorphisms of the PNMT gene and have shown a significant association of double negative heterosis (Comings and MacMurray 2000) at these two SNPs with EOAD (Mann et al. 2001). Both of these polymorphisms were examined in this study. PNMT We utilized two common SNPs from the promoter region of the PNMT gene. These were originally termed G->A-148 and G->A-353 (Wu and Comings 2000; Mann et al. 2001). This nomenclature has been updated to G->A-182 and G->A-387 for Accession #J03280. The amplified products were digested by Mnl I and Msp I restriction enzymes, for G-387A and G-181A respectively. The analysis was performed using the conditions previously described (Wu and Comings 2000).

The present invention provides diagnostic and prognostic methods for detecting LOAD using the above identified genes and alleles. It will readily be appreciated by one skilled in the art that the methods of the present invention can further be utilized to identify additional genes and/or alleles of the presently disclosed genes associated with LOAD. That is, while the present examples demonstrate the identity of AD-risk associated alleles and genes and LOAD-risk associated alleles and genes, it is considered within the scope of the present invention to apply the present methods to determine other genes which may contribute to polygenic AD and/or LOAD cases.

Once a set of candidate genes and alleles has been identified using the methods of the present invention, markers for said genes and alleles can be utilized in diagnostic determination of a risk for a disease or disorder. Useful diagnostic techniques include, but are not limited to fluorescent in situ hybridization (FISH), direct DNA sequencing, PFGE analysis, Southern blot analysis, single stranded conformation analysis (SSCA), RNase protection assay, allele-specific oligonucleotide (ASO), nested PCR followed by restriction enzyme digestion, dot blot analysis and PCR-SSCP. Also useful are techniques employing DNA microchip technology.

Predisposition to Alzheimer's disease can be ascertained by testing any tissue of a human for mutations of one or more candidate genes and/or alleles. The presence of an allele can be determined by testing DNA from any tissue of the person's body. Most simply, blood can be drawn and DNA extracted from the cells of the blood. In addition, prenatal diagnosis can be accomplished by testing fetal cells, placental cells or amniotic cells for polymorphisms.

There are several methods well known to persons of ordinary skill in the art that can be used to detect DNA sequence variation, including direct DNA sequencing, clamped denaturing gel electrophoresis, heteroduplex analysis and chemical mismatch cleavage. An allele-specific detection approach such as allele-specific oligonucleotide (ASO) hybridization can be utilized to rapidly screen large numbers of other samples for candidate genes and/or alleles.

Detection of point mutations can be accomplished by molecular cloning of the allele(s) and sequencing the allele(s) using techniques well known to persons of ordinary skill in the art. Alternatively, the gene sequences can be amplified directly from a genomic DNA preparation using known techniques. The DNA sequence of the amplified sequences then can be determined directly or with restriction enzyme analysis to detect polymorphic sites.

DNA sequences of a gene which have been amplified by use of PCR may also be screened using allele-specific oligomer probes, each of which contains a region of the gene sequence harboring a known mutation. For example, one oligomer may be about 30 nucleotides in length (although shorter and longer oligomers can be used, as recognized by those of ordinary skill in the art), corresponding to a portion of the gene sequence. By use of a battery of such allele-specific probes, PCR amplification products can be screened to identify the presence in an individual of an allele. Hybridization of allele-specific probes with nucleic acids amplified from cells can be performed, for example, on a nylon filter. Hybridization to a particular probe under high stringency hybridization conditions indicates the presence of the same mutation in the cells as in the allele-specific probe.

Nucleic acid analysis via microchip technology is also applicable to the present invention. In this technique, literally thousands of distinct oligonucleotide probes can be applied in an array on a silicon chip. A nucleic acid to be analyzed is fluorescently labeled and hybridized to the probes on the chip. It is also possible to study nucleic acid-protein interactions using these nucleic acid microchips. Using this technique one can determine the presence of mutations, sequence the nucleic acid being analyzed, or measure expression levels of a gene of interest. The method is one of parallel processing of many, even thousands, of probes at once and can tremendously increase the rate of analysis.

Alteration of mRNA transcription can be detected by any techniques known to persons of ordinary skill in the art. These include Northern blot analysis, PCR amplification and RNase protection. Diminished mRNA transcription indicates an alteration of the wild-type gene.

Polymorphisms in a gene also can be detected by screening for alteration of the protein. For example, monoclonal antibodies immunoreactive with an allele can be used to screen a tissue. Lack of cognate antigen would indicate absence of an allele. Antibodies specific for products of an allele also could be used to detect the product of the allele. Such immunological assays can be done in any convenient format known in the art. These include Western blots, immunohistochemical assays and ELISA assays. Any means for detecting an altered protein can be used to detect polymorphisms of gene. Functional assays, such as protein binding determinations, also can be used. In addition, assays which detect biochemical function can be used.

The diagnostic method of the present invention is useful to clinicians for aiding decisions as to an appropriate course(s) of treatment. It is also contemplated by the present invention that determination of heterozygosity versus homozygosity will further aid in diagnosis of Alzheimer's disease.

Primer pairs specific for a gene or allele are useful for determination of the nucleotide sequence of a particular allele using PCR. The pairs of single-stranded DNA primers can be annealed to sequences within or surrounding a gene in order to prime amplifying DNA synthesis of the gene itself. Allele-specific primers also can be used. Such primers anneal only to particular ACP1 alleles, and thus will only amplify a product in the presence of the particular allele as a template. In one embodiment, the allele-specific primers will amplify a nucleic acid comprising a particular allele but not other allelic variants.

In order to facilitate subsequent cloning of amplified sequences, primers may have restriction enzyme site sequences appended to their 5′ ends. Thus, all nucleotides of the primers are derived from sequences specific for a gene or sequences adjacent to the gene, except for the few nucleotides necessary to form a restriction enzyme site. Such enzymes and sites are well known to persons of ordinary skill in the art. The primers themselves can be synthesized using techniques which are well known to persons of ordinary skill in the art. Generally, the primers can be made using oligonucleotide synthesizing machines which are commercially available.

The nucleic acid probes provided by the present invention are useful for a number of purposes. They can be used in Southern hybridization to genomic DNA and in the RNase protection method for detecting point mutations. The probes can be used to detect PCR amplification products. They may also be used to detect mismatches with a gene or mRNA using other techniques.

In order to detect a gene variant, a biological sample is prepared and analyzed for a difference between the sequence of the allele being analyzed and the sequence of other known alleles. In a preferred embodiment, the disease or disorder is LOAD and the allele detected is selected from the group consisting of the e4 allele of the APOE gene; the VNTR polymorphism of the SLC6A4 gene; the ACP1*A allele of the ACP1 gene; the C>T 22251 polymorphism of the ACE gene; the G>A-148 or G>A-353 polymorphisms of the PNMT gene; and the Xbal or PvuII polymorphisms of the ESR gene.

“Antibodies.” The present invention also provides polyclonal and/or monoclonal antibodies and fragments thereof, and immunologic binding equivalents thereof, which are capable of specifically binding to a polypeptide and fragments thereof encoded by an allele associated with AD or LOAD. The term antibody is used both to refer to a homogeneous molecular entity, or a mixture such as a serum product made up of a plurality of different molecular entities. Antibodies will be useful in assays as well as pharmaceuticals. Antibodies to an allele will particularly be useful in detecting the allele and aiding in the diagnosis of a predisposition to AD or LOAD.

An immunological response is usually assayed with an immunoassay. Normally, such immunoassays involve some purification of a source of antigen, for example, that produced by the same cells and in the same fashion as the antigen. A variety of immunoassay methods are well known by persons of ordinary skill in the art.

As used herein, the singular form “a”, “an”, and “the” include plural references unless the context clearly indicates otherwise.

As used herein, the terms “diagnosing” or “prognosing,” as used in the context of Alzheimer's disease, are used to indicate classification, severity or monitoring of the disease progression, prior to, during or after treatment. Signs and symptoms associated with Alzheimer's disease are well known by those skilled in the art and include for example neurofibrillary tangles; Alzheimer's plaques; memory loss; and decreased ability to learn or perform a task. Various models for detecting learning and memory impairments and other tests such as for example water maze tests and passive avoidance tasks are well known by those skilled in the art and can be utilized in animal models of the present invention.

Polynucleotide compositions useful in the practice of this invention include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule (Peptide Nucleic Acids or “PNA's”). The polynucleotides of the invention may be isolated or substantially pure. Oligonucleotides which detect the genes utilized in the present invention or analogues of such oligonucleotides can also be prepared. Such analogues may constitute alternative structures such as “PNA's” or the like. It is evident that these alternative structures, representing the sequences of the present invention, are likewise part of the present invention.

cDNA or genomic libraries of various types may be screened as natural sources of the nucleic acids of a particular allele, or such nucleic acids may be provided by amplification of sequences resident in genomic DNA or other natural sources, e.g., by PCR. The choice of cDNA libraries normally corresponds to a tissue source which is abundant in mRNA for the desired proteins. Phage libraries are normally preferred, but other types of libraries may be used. Clones of a library are spread onto plates, transferred to a substrate for screening, denatured and probed for the presence of desired sequences.

Polynucleotide polymorphisms associated with particular alleles from candidate genes for a disorder, including alleles which are useful for diagnosing or predicting Alzheimer's disease, can be detected by hybridization with a polynucleotide probe which forms a stable hybrid with that of the target sequence, under highly stringent to moderately stringent hybridization and wash conditions. If it is expected that the probes will be perfectly complementary to the target sequence, high stringency conditions will be used. Hybridization stringency may be lessened if some mismatching is expected, for example, if variants are expected with the result that the probe will not be completely complementary. Conditions are chosen which rule out nonspecific/adventitious bindings, that is, which minimize noise.

Nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringent temperature conditions will generally include temperatures in excess of 300° C., typically in excess of 37° C., and preferably in excess of 45° C. Stringent salt conditions will ordinarily be less than 1000 mM, typically less than 500 mM, and preferably less than 200 mM. However, the combination of parameters is much more important than the measure of any single parameter. The stringency conditions are dependent on the length of the nucleic acid and the base composition of the nucleic acid, and can be determined by techniques well known by persons of ordinary skill in the art.

The probes can include an isolated polynucleotide attached to a label or reporter molecule and may be used to isolate other polynucleotide sequences having sequence similarity, by standard methods. Other similar polynucleotides may be selected by using homologous polynucleotides. Alternatively, polynucleotides encoding these or similar polypeptides may be synthesized or selected by use of the redundancy in the genetic code. Various codon substitutions may be introduced, e.g., by silent changes (thereby producing various restriction sites) or to optimize expression for a particular system. Mutations may be introduced to modify the properties of the polypeptide, perhaps to change ligand-binding affinities, interchain affinities, or the polypeptide degradation or turnover rate.

Polypeptides comprising a particular allele, if soluble, may be coupled to a solid-phase support, e.g., nitrocellulose, nylon, column packing materials (e.g., Sepharose beads), magnetic beads, glass wool, plastic, metal, polymer gels, cells, or other substrates. Such supports may take the form, for example, of beads, wells, dipsticks, or membranes.

“Recombinant nucleic acid” is a nucleic acid which is not naturally occurring, or which is made by the artificial combination of two otherwise separated segments of sequence. This artificial combination is often accomplished by either chemical synthesis means, or by the artificial manipulation of isolated segments of nucleic acids, e.g., by genetic engineering techniques.

In order to detect the presence of a group of alleles predisposing an individual to a disease or disorder, e.g., AD or LOAD, a biological sample such as blood is prepared and analyzed for the presence or absence of predisposing alleles by analyzing the individual's genetic material. AS used herein, the analysis of genetic material may be direct, through examination of a nucleic acid, or indirect, such as by examination of an altered amino acid produced by the individual's genetic material. Such diagnoses may be performed by diagnostic laboratories, or, alternatively, diagnostic kits are manufactured and sold to health care providers or to private individuals for self-diagnosis.

Initially, the screening method may involve amplification of the relevant sequences. In another preferred embodiment of the invention, the screening method involves a non-PCR based strategy. Such screening methods can include for example two-step label amplification methodologies that are well known to persons of ordinary skill in the art. Both PCR and non-PCR based screening strategies can detect target sequences with a high level of sensitivity. As will readily be appreciated by those skilled in the art, an allele can be detected with various techniques, including for example PCR and enzymatic digestion of the TaqI site, PCR amplification with specific primers (i.e., allele-specific amplification) and or hybridization analysis of genomic DNA with a probe that specifically hybridizes to an allele (i.e., allele specific probes as oligonucleotides).

Preferred embodiments relating to methods for detecting polymorphisms include enzyme linked immunosorbent assays (ELISA), radioimmunoassays (RIA), immunoradiometric assays (IRMA) and immunoenzymatic assays (IEMA), including sandwich assays using monoclonal and/or polyclonal antibodies.

The practice of the present invention employs, unless otherwise indicated, conventional techniques of chemistry, molecular biology, microbiology, recombinant DNA, genetics, immunology, cell biology, cell culture and transgenic biology, which are within the skill of the art.

Gene codes. The three possible genotypes of each gene are assigned a score each based on the respective means of the AD score for each genotype by treating AD as a continuous trait. For example, the genotype with the lowest mean AD scored was scored as 0, and the genotype with the highest AD score was scored as 2. In a preferred embodiment, the lowest score is subtracted from the highest score to determine the difference between the two (l−h), and the lowest score is subtracted from the score for the remaining genotype (l−r). The remaining genotype is assigned a code of 0 if 1−r is less than 33% of l−h, a code of 2 if it is greater than 66% of l−h, and a code of 1 if it is between 36 and 65% of l−h. In noting the gene score for a given gene and a given phenotype, the score for the 11 genotype is given first, that for the 12 genotype is given second, and that for the 22 genotype is given last. As discussed in more detail elsewhere (Comings et al. 2000b) this identifies 12 types of inheritance. For example, a score of 012 would represent a 2 allele co-dominant mode of inheritance. A 202 score would represent a negative heterosis mode of inheritance (Comings and MacMurray 2000). If six genes were examined and an individual carried all of the genotypes least associated with a given phenotype their total score would be 0. If they carried all the genotypes most associated with the phenotype their score would be 12.

Statistics. Alleles and genotypes were counted and their distributions between groups were determined by the Chi-square (χ²) test. Differences were considered significant at the p≦0.05 level. To evaluate the direction of association of each individual gene with the AD score, we treated the AD OR LOAD score as a continuous trait, and the mean AD OR LOAD score for each genotype of each gene was assessed using ANOVA. The Chi-square and ANOVA tests were performed using the SPSS statistical package for the Macintosh (release 6.1.1) (SPSS, Inc, Chicago, Ill.).

The relative r, r², p values and odds ratio for genes when tested either singly or together were determined by logistic regression analysis using the SAS statistical package (SAS Institute, Inc, Cary, N.C.). The AD OR LOAD score was the dependent variable and the gene scores (0, 1 or 2) were the independent variables. For multivariate logistic regression analyses, we applied the stepwise procedure to select the model with the entry value set at 0.2 and the stay value set at 0.7. Since the SAS program for multivariate logistic regression did not give r² values for each gene, these were determined by subtraction of the cumulative total r² values for the previous step from the current step. The use of regression analysis mirrors the technique used in our prior studies of the additive effects of multiple genes (Comings et al. 2000a,b,c; 2001).

Weighted gene scores. Since the different genes contributed to a markedly different percent of the variance of the LOAD score, in one of the analyses each gene score was weighted on the basis of the r² value for that gene as determined by logistic regression analysis. This value was then multiplied by 10 and used as the weighting factor.

To evaluate possible population stratification, and evaluate whether our sample size could cause unstable variation in model selection, we applied a bootstrap simulation procedure for this bias. A total of 1000 replicates, each containing 500 sample size drawn from the original dataset with replacement, were simulated. A standard error was estimated from the bootstrap samples and used to calculate corrected 95% confidence interval of odds ration for the significant genes. All these procedures were performed by STATA (V. 7.0, STATA Corp., TX).

ROC plots. Receiver-Operator-Characteristic (ROC) plots provide a pure index of the accuracy of a given test by demonstrating the limits of the test's ability to discriminate between alternative states of health or disease over the complete spectrum of operating conditions (Zweig and Campbell 1993; Metz 1978). The ROC plot depicts the overlap between the two distributions by plotting the sensitivity (Y-axis) versus (1-specificity) (X-axis) for the complete range of decision thresholds, where sensitivity, or the true-positive fraction, is defined as (number of true-positive test results)/(number of true-positive+number of false-positive test results) and referred to as positivity in the presence of a disease based on calculations for the affected group. Specificity is defined as (number of false-positive results)/(number of true-negative+number of false-positive results), an index of true negative, and is calculated from the unaffected group (Zweig and Campbell 1993) (Griner et al. 1981).

Computer programs enhance the ease of use of ROC curves (Zweig and Campbell 1993). These allow the determination of the positive and negative likelihood ratios for the presence of disease for each of the sensitivity-specificity pairs. The program also calculates the area under the curve, a further measure of the effectiveness of the test (Hanley and McNeil 1982). The ROC plots, likelihood ratios and area under the curve were calculated using the MedCalc version 6.11.001 (Mariakerke, Belgium).

Calculation of risk for each PG score. In the ROC output, the positive likelihood ratios have a value of 1 for the individuals with the lowest risk score and progressively higher relative scores for individuals with progressively higher risk scores. The negative likelihood ratios have a value of 1 for the individuals with the highest risk score and progressively increase for individuals with progressively lower scores. Since a score of 1 for individuals with a neutral risk, and a score of 0 to 1 for individuals with lower risk, and a score of 1 or more for individuals with a higher risk, is easier to interpret, we multiplied the positive likelihood risk by the negative likelihood risk to produce a score we have simply termed the likelihood risk. We examined an alternative technique in which an odds ratio was obtained by dividing the frequency of a given PG score in subjects by the frequency in controls. Since both techniques give similar results we have only used the likelihood risk approach. The one exception is when the negative likelihood risk is 0. Since division by 0 is not possible is this case the odds ratio can be substituted.

### EXAMPLES

The present invention is further detailed in the following Examples, which are offered by way of illustration and are not intended to limit the invention in any manner. Standard techniques well known in the art or the techniques specifically described below, or in are utilized.

### Example I

Examination of Individual Genes with LOAD. The comparison of set 1 and set 2 is shown in Table 1. There were a total of 296 controls and 204 LOAD subject, with 250 individuals in each set for a total of 500 individuals in the study. The number of controls and LOAD cases in the two sets was identical and the mean age and sex ratio for the two sets was virtually identical. Although the mean age of the LOAD subjects was greater than that of the controls, since our LOAD subjects came from a brain bank their age is their age at death. By contrast the controls are still living. We suspect that if the controls were followed until death and that was used instead of current age, the ages of the two samples would be more similar. However, some of the controls could have developed LOAD during that time and our results (see below) are consistent with the presence of some potential LOAD cases in the control group.

The results of the association of each gene with LOAD in set 1, set 2 and the total of both sets, is shown in Table 2. First, for each gene we have first presented the results of the chi square analysis (number and percent of subjects with each genotype for controls and LOAD subjects, the chi square and the p value). Second, we present the gene code (see methods) for both sets and for the total set. Third, we present the results of the logistic regression analysis between the gene score and the AD score. These results are given as r², or fraction of the variance of the phenotype explained, the p value of this correlation, and the odds ratio.

APOE. The APOE genotypes were grouped into non4/non4, non4/4, and 4/4. As is typical of the literature on APOE, there was a significant increase in the frequency of the non4/4 and 4/4 genotypes in individuals with AD. The gene scores were similar but not identical for the two sets. For set 1 it was 021 indicating the AD score was higher for those with the non4/4 genotype than for the 4/4 genotype. For set 2 and the total of both sets the gene score was 012 indicating the AD score was progressively higher across the three genotypes. For the total set 23.6% of the controls versus 52.5% of the LOAD subjects carried the non4/4 genotype. The respective figures were 2.7% and 12.3% for the 4/4 genotype, p≦0.0001. The chi square for set 1 was 50.60 and for set 2 was 30.63 and both were significant at p≦0.00001. By logistic regression analysis the r² was 0.187 for set 1 and 0.117 for set 2, indicating that the APOE gene accounted for considerably more of the variance in set 1 compared to set 2. Thus, while there was some minor variability in the gene scores between the two sets and even though the r² for set 1 was substantially higher than for set 2, the results were strongly significant for both sets.

ACE, DCP1. Our pilot studies showed a significant association between the ACE C/T 22251 polymorphism and LOAD. By chi square analysis the association of the C/T 22251 polymorphism and LOAD was significant for both sets, p≦0.042 and p≦0.020 respectively and p≦0.0016 for both sets. Based on the means of the AD score, the ACE gene was scored as 11=2, 12=0 and 22=1, abbreviated as 201 for set 1, set 2 both sets together. This is indicative of negative heterosis. The r² for set 1 was 0.0231, p≦0.0156, for set 2 was 0.0276, p≦0.0082 and for the total group r² was 0.0253, p≦0.0003. The odds ratios for all three groups were 1.458 or greater. Thus, for the ACE gene the gene scores were the same for both sets, the association with LOAD was significant for both sets by chi square analysis and by gene score-phenotype regression analysis, and the odds ratios were similar for both.

ACP1. By chi square analysis there was no significant association between gene and LOAD for either set or for the total group. The gene codes for both sets and the total of both sets were also different. Finally, despite using their respective gene codes the r² values were also non-significant for both sets and the total group and the odds ratios were all less than 1.23. The odds ratio for the total of both sets was not intermediate between the individual sets because the gene code (022) was different than for either set 1 (020) or set 2 (012). In summary, for this gene, despite some suggestive pilot studies showing an association with EOAD, the results were negative in this study of LOAD.

ESR1 Xba I. By chi square analysis the association with LOAD was significant for both set 1 (p≦0.0076) and 2 (p≦0.00084), and the total group (p≦0.00005). The gene codes for the three groups were 002, 202, and 102. Based on the gene scores, r² was 0.0378 for set 1, 0.0548 for set 2, and 0.0398 for both sets. All were significant at p≦0.0019. Thus, based on the Xba I polymorphism, the ESR1 gene was significantly associated with LOAD in both sets.

ESR1 Pvu II. By chi square analysis the association of the Pvu II with LOAD was significant in set 1 (p≦0.0074), non-significant in set 2, and significant for the total group (p≦0037). The gene score of 102 was the same for all three groups and indicative of negative heterosis, analogous to the results for the Xba I polymorphism. The r² value was significant for set 1 (0.0363, p≦0.0024) and the total group (0.0214, p≦0.001) but not for set 2 (0.0102, p≦0.1090). The r² for both sets together for ESR1 Pvu II (0.0214) was almost half of that for the Xba I polymorphism (0.0389).

Since both of the gene scores for the Xba T and the Pvu II polymorphisms were suggestive of negative heterosis we sought to determine if the simultaneous examination of both polymorphisms in terms of homozygosity versus heterozygosity would give more significant results than either polymorphism alone. A 3 by 3 table (Xba I genotypes 11, 12, 22 by Pvu II genotypes 11, 12, 22) produced 9 possible groups for each individual. We first tested whether the mean AD score was significantly different by a 9 way ANOVA. Both sets and the total group were significant at p≦0.007. We then collapsed the 9 groups into 3 groups of homozygote Xba I homozygote Pvu II, homozygote/heterozygote, and heterozygote Xba I/heterozygote Pvu II. When examined by chi square analysis both sets and the total group were significant at p≦0.0054 (data not shown). The gene score for the combined polymorphisms was 220 for all three sets, where position 1=the homozygous×homozygous group, position 2=the homozygous by heterozygous group, and position 3=the double heterozygotes. Using this gene code, the r² values were significant for set 1 (r²=0.0426, p≦0.001), for set 2 (r²=0.0524, p≦0.0002), and for both sets together (r²=0.0469, p≦0.0001). When both polymorphisms were used, the r² for the total of both sets was higher (0.0469) than for the XbaI polymorphism alone (0.0389). However, since this was not more than the sum of the two independent r² values (0.0389+0.0214=0.0603), for subsequent analyses and ROC studies we used the two polymorphisms independently.

PNMT G->A-182. By chi square analysis, neither set 1 (p≦0.884) nor set 2 (p≦0.944) showed a significant correlation between the PNMT G->A-182 polymorphism and LOAD. The gene codes were different for set 1 (002) and set 2 (020) and both sets (022). These are not accurate when the p values are so high. Based on the respective gene scores the r² values were low and also non-significant. Thus, on the basis of just the G->A-182 polymorphism, the PNMT gene appeared to play no role in the risk for LOAD.

PNMT G->A-387. By chi square analysis the results with the G->A-387 polymorphism were not significant for set 1, set 2 or the total group. The gene code for set 1 was 120 and 021 for set 2 and the total group. All three were indicative of positive heterosis. Using these gene scores, the r² value of 0.0191 was significant for set 2 (p≦0.028). The r² values were not significant or set 1 or the total of both sets.

As with the two ESR1 polymorphisms, we sought to determine if the effect of examining the two PNMT polymorphisms together provided more power, as it did in our studies of EOAD (Mann et al. 2001). We again produced nine different PNMT G->A-182×PNMT G->A-387 genotype combinations. The ANOVA between the LOAD score and these 9 groups was not significant for either set or the total group. To explore the role of double heterosis at the PNMT polymorphisms, the 9 groups were reduced to 3, based on homozygote/homozygote, homozygote/heterozygote and heterozygote/heterozygote sets. The gene scores for these (see ESR1 results for method of scoring) were 202 for set 1, 012 for set 2, and 102 for both sets combined. None of the resultant r² values were significant.

SLC6A4. The chi square analysis was non-significant for both sets and borderline for the total group (p≦0.056). The gene codes (022) were the same for both sets and the total group. Based on the gene scores the r² values were significant for set 2 (p≦0.0455) and for the total group (p≦0.0179).

Multivariate Logistic Regression analysis. We simultaneously examined the genes/polymorphisms in a multivariate logistic regression analysis in the presence (Table 3A) and absence (Table 3B) of the APOE gene. For all genes, with set 1 the included genes/polymorphisms, in order of r² values were APOE, ESR1 Xba I, ACP1, ESR1 PvuII, ACE and SLC6A4. The sum of the individual r² values was 0.2407. The p value for the total based on maximum likelihood chi square was ≦0.0001. The odds ratio was 2.64 (bootstrap bias-corrected 95% CI=2.2-7.7) for the APOE gene; 1.38 (bias-corrected 95% CI=0.98-1.99 from bootstrap) for ESR1 XbaI.

The weights (see methods) are also given in Table 3A. For set 1 these were 7.79 for the APOE gene compared to ≧0.77 for the remaining genes. Thus, the APOE gene was weighted 10.1 times or more than the other genes in set 1.

For set 2, the included genes/polymorphism in order of r² values were APOE, ESR1 Xba I, ACE, and PNMT G->A-387 and SLC6A4. The summed r² was 0.2051, p≦0.0001. The odds ratio was 3.15 (bias-corrected 95% CI=1.9-4.8 from bootstrap) for the APOE gene; 1.67 (bias-corrected 95% CI=1.2-2.7 from bootstrap) for ESR Xba I; 1.57 (95% CI=1.0-2.25 from bootstrap) for ACE; 1.52 (95% CI=0.95-2.4, p>0.05 from bootstrap) for PNMT G->A -387.

The weights were 5.70 for APOE gene compared to ≧1.94 for the remaining genes. Thus, compared to the other genes the APOE gene was scored only 2.9 fold or more. Both sets included the APOE, ESR1 Xba I, ACE and SLC6A4 genes. The ESR1 Pvu II gene was included in set 1 but not set 2, while the PNMT G->A-387 gene was included in set 2 but not set 1. The summed r² was similar for both sets, 0.2407 and 0.2051. When both sets were combined the APOE, ESR1 Xba I, ACE, and SLC6A, PNMT G->A-38, ESR1 Pvu II and ACP1 genes were included. The summed r² was 0.1976, p≦0.0001.

The total r² for the four genes exclusive of the APOE gene was also examined (Table 3B). For set 1 the included genes in order of their r² values were ESR1 Xba I, ACE, and ESR1 Pvu II. The r² for the combined set was 0.791, p≦0.0001. For set 2, the included genes were ESR1 Xba I, ACE, PNMT G>A-387 and SLC6A4. Combined they produced a summed r² of 0.1179 p≦0.0001. For set 1+set 2 combined the included genes were ESR1 Xba I, ACE, SLC6A4, PNMT G>A-387 and ESR1 Pvu II. Combined they produced a summed r² of 0.0837, p≦0.0001.

LOAD Risk Scores. For the third step we examined several methods of forming a PG score, including APOE gene alone, all other genes, all genes together, and weighted all genes.

Comparison of the PG risk scores by ROC plots. This step was to examine these different PG scores in a ROC plot. For comparative purposes, for a given score the results for set 1 and set 2 were examined in the same plot. Since the plot for set 1 and set 2 were approximately intermediate between the two, the plots for set 1+set 2 are not shown.

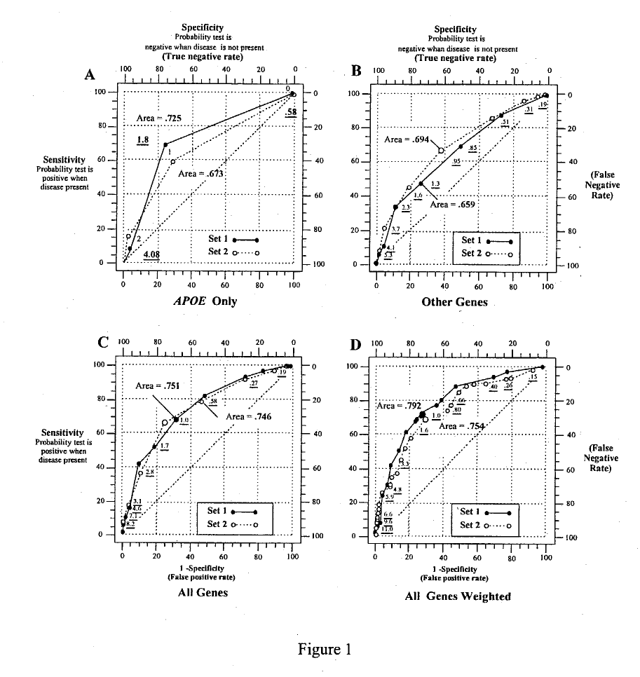

The plots for the APOE gene alone are shown in FIG. 1A. Since there were only 3 scores, 0=non-4/non4, 1=non-4/4, and 3=4/4, there were only 3 points on the plots. The area under the curve for set 1=0.725 and for set 2=0.673. For set 1+2 it was 0.70. The relative risk scores for set 1 (which showed the greatest influence of the APOE genes), are shown in FIG. 1A (underlined). These were 1.8 for a score of 1 and 4.08 for a score of 2. The score of 0 had a negative relative risk of 0. To avoid the problem of 0 negative relative risk scores (see methods); the odds ratio risk of 0.58 was used for the score of 0.

The ROC plots for all other included genes except APOE for set 1 and 2 from Table 3B are shown in FIG. 1B. Even though different genes were involved in the two sets, the area under the curve was the similar for both, 0.694 for set 1 and 0.659 for set 2. For the two sets the relative risks (underlined) of each score ranged from 0.19 to 5.3

The ROC plots for all of the included genes from Table 3A are shown in FIG. 1C. The area under the curve was similar for both: 0.751 for set 1 and 0.746 for set 2. The average relative risks for the different scores ranged from 0.19 to 8.2.

The results of the ROC plots for the weighted gene scores for set 1 and set 2 are shown in FIG. 1D. The areas under the curves were 0.792 for set 1, 0.754 for set 2. The relative risk scores for each gene scores are underlined. They ranged from a low of 0.15 to a high of 11.0.

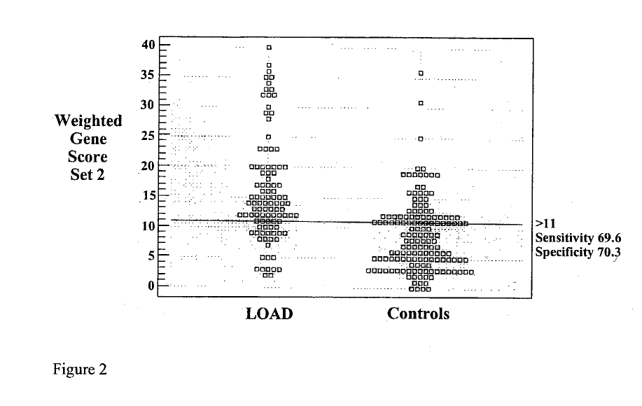

FIG. 2 shows the cutpoint where the sensitivity and specificity are maximized. At a break point of 11, 69.6% of the LOAD cases had a score of greater than 11 and 70.3% of the controls had a score of 11 or less. Interestingly, there were three control subjects with a weighted score of 20 or more, compared to 16 LOAD subjects with this score. These three controls could represent individuals who will develop LOAD as they get older.

TABLE 1
Comparison of Set 1 and Set 2.

| | Set 1 | Set 2 | Total |
|---|---|---|---|
| A. Diagnoses | N (%) | N (%) | |
| Controls | 148 (50.0) | 148 (50.0) | 296 |
| LOAD | 102 (34.5) | 102 (34.5) | 204 |
| Total | 250 | 250 | 500 |
| B. Age | Mean (S.D.) | Mean (S.D.) | |
| Controls | 66.30 (8.12) | 66.10 (7.75) | |
| F-ratio | .042 | | |
| p | .838 | | |
| LOAD | 81.65 (6.63) | 81.44 (6.37) | |
| F-ratio | .0561 | | |
| p | .813 | | |
| C. Sex | N M/F (% male) | N M/F (% male) | |
| Controls | 91/57(61.5) | 90/58 (60.8) | |
| Chi-square | .014 | | |
| p | .905 | | |
| LOAD | 46/56 (45.1) | 45/57 (44.1) | |
| Chi-square | .019 | | |
| p | .88 | | |

TABLE 2
Association of Individual Genes with LOAD for Sets 1 and 2 and Total

| | Set 1 | | Set 2 | | Total | |
|---|---|---|---|---|---|---|
| Gene | Controls | AD | Controls | AD | Controls | AD |
| APOE | | | | | | |
| non4/non4 | 112 (75.7) | 31 (30.4) | 106 (71.6) | 41 (40.2) | 218 (65.7) | 72 (35.3) |
| non4/4 | 31 (20.9) | 62 (60.8) | 39 (26.4) | 45 (44.1) | 70 (23.6) | 107 (52.5) |
| 4/4 | 5 (3.4) | 9 (8.8) | 3 (2.0) | 16 (15.7) | 8 (2.7) | 25 (12.3) |
| χ² | 50.60 | | 30.63 | | 75.63 | |
| p | <.00001 | | <.00001 | | <.00001 | |
| gene code | 021 | | 012 | | 012 | |
| r² | .187 | | .117 | | .1387 | |
| p* | <.0001 | | <.0001 | | <.0001 | |
| O.R. | 2.669 | | 3.287 | | 3.883 | |
| ACE, DCP1 | | | | | | |
| 11 | 28 (18.9) | 33 (32.4) | 32 (21.6) | 33 (32.4) | 60 (20.3) | 66 (32.4) |
| 12 | 82 (55.4) | 44 (43.1) | 86 (58.1) | 41 (40.2) | 168 (56.8) | 85 (41.7) |
| 22 | 38 (25.7) | 25 (24.5) | 30 (20.3) | 28 (27.5) | 68 (23.0) | 53 (26.0) |
| χ² | 6.30 | | 7.83 | | 12.88 | |
| p | .042 | | .020 | | .0016 | |
| gene code | 201 | | 201 | | 201 | |
| r² | .0231 | | .0276 | | .0253 | |
| p | .0156 | | .0082 | | .0003 | |
| O.R. | 1.458 | | 1.500 | | 1.479 | |
| ACP1 | | | | | | |
| non*A/non*A | 71 (48.0) | 43 (42.2) | 79 (53.4) | 52 (51.0) | 150 (50.7) | 95 (46.62) |
| non*A/*A | 63 (42.6) | 50 (49.0) | 58 (39.2) | 39 (38.2) | 121 (40.9) | 89 (43.6) |
| *A/*A | 14 (9.5) | 9 (8.8) | 11 (7.4) | 11 (10.8) | 25 (8.4) | 20 (9.8) |
| χ² | 1.03 | | .851 | | .880 | |
| p | .597 | | .657 | | .644 | |
| gene code | 020 | | 012 | | 022 | |
| r² | .0040 | | .0033 | | .0016 | |
| p | .314 | | .362 | | .367 | |
| O.R. | 1.139 | | 1.227 | | 1.086 | |
| ESR1 Xba I | | | | | | |
| 11 | 44 (29.7) | 24 (23.5) | 28 (18.9) | 30 (29.4) | 72 (24.3) | 54 (26.5) |
| 12 | 74 (50.0) | 39 (38.2) | 95 (64.2) | 41 (40.2) | 169 (57.1) | 80 (39.2) |
| 22 | 30 (20.3) | 30 (38.2) | 25 (16.9) | 31 (30.4) | 55 (18.6) | 70 (34.3) |
| χ² | 9.76 | | 14.16 | | 19.93 | |
| p | .0076 | | .00084 | | .00005 | |
| gene code | 002 | | 202 | | 102 | |
| r² | .0378 | | .0548 | | .0389 | |
| p | .0019 | | .0002 | | <.0001 | |
| O.R. | 1.560 | | 1.633 | | 1.635 | |
| ESR1 Pvu II | | | | | | |
| 11 | 40 (27.0) | 27 (26.5) | 31 (20.9) | 24 (23.5) | 71 (24.0) | 51 (25.0) |
| 12 | 84 (56.8) | 42 (41.2) | 95 (64.2) | 56 (54.9) | 179 (60.5) | 98 (48.0) |
| 22 | 24 (16.2) | 44 (29.7) | 22 (14.9) | 22 (21.6) | 46 (15.5) | 55 (27.0) |
| χ² | 9.81 | | 2.58 | | 11.21 | |
| p | .0074 | | .274 | | .0037 | |
| gene code | 102 | | 102 | | 102 | |
| r² | .0363 | | .0102 | | .0214 | |
| p | .0024 | | .1090 | | .0010 | |
| O.R. | 1.624 | | 1.304 | | 1.456 | |

ESR1 Xba I and Pvu II

| | | | |
|---|---|---|---|
| ANOVA | 3.91 | 2.71 | 3.92 |
| F-ratio | | | |
| p | .0002 | .0070 | .0002 |
| gene score | 220 | 220 | 220 |
| r² | .0426 | .0524 | .0469 |
| p | .0010 | .0002 | <.0001 |
| O.R. | 1.637 | 1.644 | 1.629 |

PNMT G->A-182

| | | | | | | |
|---|---|---|---|---|---|---|
| 11 | 48 (32.4) | 32 (31.4) | 46 (31.1) | 27 (26.5) | 94 (31.8) | 59 (28.9) |
| 12 | 75 (50.7) | 48 (47.1) | 68 (45.9) | 53 (52.0) | 143 (48.3) | 101 (49.5) |
| 22 | 25 (16.9) | 22 (21.6) | 34 (23.0) | 22 (21.6) | 59 (19.9) | 44 (21.6) |
| χ² | .884 | | .944 | | .510 | |
| p | .643 | | .923 | | .775 | |
| gene code | 002 | | 020 | | 022 | |
| r² | .0034 | | .0035 | | .0009 | |
| p | .354 | | .350 | | .498 | |
| O.R. | 1.163 | | 1.128 | | 1.069 | |

PNMT G->A-387

| | | | | | | |
|---|---|---|---|---|---|---|
| 11 | 28 (18.9) | 20 (19.6) | 45 (30.4) | 20 (19.6) | 73 (24.7) | 40 (19.6) |
| 12 | 65 (43.9) | 50 (49.0) | 51 (34.5) | 47 (46.1) | 116 (39.2) | 97 (47.5) |
| 22 | 55 (37.2) | 32 (31.4) | 52 (35.1) | 35 (34.3) | 107 (36.1) | 67 (32.8) |
| χ² | .94 | | 4.79 | | 3.72 | |
| p | .62 | | .091 | | .155 | |
| gene code | 120 | | 021 | | 021 | |
| r² | .0036 | | .0191 | | .0071 | |
| p | .342 | | .028 | | .0587 | |
| O.R. | 1.147 | | 1.433 | | 1.248 | |

| | |
|---|---|
| PNMT -182 and -387 | 3 × 3 = 9 variables |

| | | | | | | |
|---|---|---|---|---|---|---|
| ANOVA | 1.06 | | 1.63 | | .877 | |
| F-ratio | | | | | | |
| p | .394 | | .115 | | .535 | |
| gene code | 202 | | 012 | | 102 | |
| r² | .0094 | | .0105 | | .0055 | |
| p | .1238 | | .1036 | | .0972 | |
| O.R. | 1.249 | | 1.283 | | 1.216 | |
| SLC6A4 | | | | | | |
| non12/non12 | 25 (16.9) | 11 (10.8) | 24 (16.2) | 8 (7.8) | 49 (16.6) | 19 (9.3) |
| heterozygous | 62 (41.9) | 46 (45.1) | 72 (48.6) | 49 (48.0) | 134 (45.3) | 95 (46.6) |
| 12/12 | 61 (41.2) | 45 (44.1) | 52 (35.1) | 45 (44.1) | 113 (38.2) | 90 (41.1) |
| χ² | 1.82 | | 4.56 | | 5.75 | |
| p | .40 | | .102 | | .056 | |
| gene code | 022 | | 022 | | 022 | |
| r² | .0075 | | .0159 | | .0111 | |
| p | .170 | | .0455 | | .0179 | |
| O.R. | 1.297 | | 1.508 | | 1.390 | |

p values based on likelihood ratio chi square

TABLE 3
Results of Multivariate Logistic Regression Analyses

| Gene | r² | p | O.R. | weights |
|---|---|---|---|---|
| A. All Genes | | | | |
| Set 1 | | | | |
| APOE | .1874 | .0001 | 2.644 | 7.79 |
| ESR1 Xba 1 | .0186 | .0155 | 1.368 | 0.77 |
| ACP1 | .0112 | .0596 | 1.301 | 0.47 |
| ESR1 Pvu II | .0098 | .0767 | 1.417 | 0.41 |
| ACE | .0082 | .1007 | 1.357 | 0.34 |
| SLC6A4 | .0055 | .1867 | 1.339 | 0.23 |
| Sum r² | .2407 | <.0001 | | |
| Set 2 | | | | |
| APOE | .1170 | .0001 | 3.155 | 5.70 |
| ESR Xba 1 | .0397 | .0007 | 1.670 | 1.94 |
| ACE | .0204 | .0133 | 1.569 | 0.99 |
| PNMT G->A-387 | .0183 | .0184 | 1.520 | 0.89 |
| SLC6A4 | .0097 | .0891 | 1.517 | 0.47 |
| Sum r² | .2051 | <.0001 | | |
| Sets 1 and 2 | | | | |
| APOE | .1387 | <.00001 | 3.754 | 7.02 |
| ESR1 Xba 1 | .0239 | .0002 | 1.476 | 1.21 |
| ACE | .0147 | .0028 | 1.420 | 0.74 |
| SLC6A4 | .0103 | .0144 | 1.474 | 0.52 |
| PNMT G->A-387 | .0039 | .1208 | 1.228 | 0.20 |
| ESR1 Pvu II | .0031 | .1639 | 1.206 | 0.16 |
| ACP1 | .0030 | .1685 | 1.159 | 0.15 |
| Sum r² | .1976 | <.0001 | | |
| B. All genes except APOE. | | | | |
| Set 1 | | | | |
| ESR1 Xba 1 | .0389 | .0018 | 1.467 | |
| ACE | .0246 | .0110 | 1.470 | |
| ESRi Pvu II | .0167 | .0335 | 1.432 | |
| Sum r² | .0791 | <.0001 | | |
| Set 2 | | | | |
| ESR1 Xba I | .0548 | .0002 | 1.701 | |
| ACE | .0282 | .0059 | 1.601 | |
| PNMT G->A-387 | .0222 | .0139 | 1.509 | |
| SLC6A4 | .0127 | .0648 | 1.514 | |
| Sum r² | .1179 | <.0001 | | |
| Set 1 + 2 | | | | |
| ESR1 Xba I | .0389 | <.0001 | 1.542 | |
| ACE | .0261 | .0002 | 1.506 | |
| SLC6A4 | .0088 | .0324 | 1.366 | |
| PNMT G->A-387 | .0057 | .0810 | 1.227 | |
| ESR Pvu II | .0042 | .1288 | 1.210 | |
| Sum r² | .0837 | <.0001 | | |

### Example II

Ad Scores

AD+control samples were genotyped at APOE, ESR1, SLC6A4, ACE and ACP1A. The genotypes were placed into three genotype groups. For bi-allelic SNPs these groups were 11, 12 and 22. For the APOE e2-4 polymorphism the genotypes consisted of 4/4 homozygotes, 4/other heterozygotes, and non4/non4 subjects. For the SLC6A4 sertrans polymorphism the genotypes were 12/12 homozygotes, 12/non12 heterozygotes, and non12/non12 homozygotes. For the remaining genes the three genotypes were 11, 12 and 22. The three genotypes of each gene/polymorphism set were examined by ANOVA against an AD score, which was scored as controls=0 and AD cases=1. These results are shown in Table 4 and are presented several ways. First, the mean AD score and standard deviation for each genotype are given. Second the F-ratio and p value for the ANOVA for the three genotypes are shown. Third, an F-ratio (F_{L}-ratio) and p value (P_{L} is given for the linear ANOVA results. If the given gene/polymorphism shows a semi-dominant or co-dominant mode of inheritance with the AD scores progressively increasing or decreasing across the three genotypes such that 22>12>11 and the F_{L}-ratio and the P_{L} will be greater than the standard F-ratio and p value. By contrast, if the mode of inheritance shows heterosis, such that the AD score for the 12 heterozygotes are greater (or lesser) than for both homozygotes, the standard F-ratio and p values will be greater than for the linear F-ratio and linear p value. The results are also examined by regression analysis. This gives the correlation, r, between the AD score and the genotype scores and the fraction of the variance, r², and significance level, p. For each gene, the r and r² were maximized by assigning a gene score such that the genotype with the lowest AD score=0, the genotype with the highest AD score=2 and the remaining genotype was assigned a value of 0, 1 or 2 depending upon whether it was closer to the lowest, highest or an intermediate value. These results are also given in Table 4. Table 4 shows that the APOE gene and the e2-4 polymorphism is the most strongly associated with the AD score of the five genes in the panel. Thus, the F-ratio is 39.7, p<0.0001 compared to F-ratios of 3.30 to 8.0 and p values of 0.0004 to 0.035 for the other four genes. In addition, the F_{L}-ratio was 78.79 and the p value<0.0001 for the APOE gene compared to F_{L}-ratios of 1.29 to 6.97 and p values of 0.0086 to 0.255 for the other four genes. By regression analysis, the r² value for the APOE gene was 0.16, p<0.0001 compared to 0.014 to 0.035 and p value of 0.011 to 0.0001 for the other four genes.

Multivariate regression analysis. To examine the correlation between the five genes in the panel and AD, the gene scores were examined simultaneously by multivariate regression analysis. Based on the ˜values of the individual genes, the APOE gene was 4.61 times more powerful than the highest ˜value for the other genes (0.162/0.035=4.61). To adjust for this the gene score for the APOE gene was increased by 5 fold such that 4/4 homozygotes=10 and 4/other heterozygotes=5. This had no effect on the r² value for APOE alone which was still 0.162. The resulting APOE gene score and the other four gene scores were entered into a multivariate regression analysis using backward elimination. The results, shown in Table 5, are placed in descending order by the T values. These were respectively APOE-8.919, SLC6A4-3.687, ESR13.547, ACP 1 A-2.490, and ACE 2.111. Other than the APOE gene, the p values range from 0.0003 for the SLC6A4 gene to 0.035 for the ACE gene. The multiple r for all five genes was 0.49, and r²=0.242, and adjusted r²=0.233. Thus that addition of the four non-APOE genes resulted in an increase in the percent of the variance explained from 0.16 for the APOE gene alone, to 0.24 for the whole panel of five genes. To validate this independently of the APOE gene, the four non-APOE genes were included in a multivariate regression analysis. These results are shown in Table 6. Although the order of the genes was somewhat different the results were similar. Thus the T values ranged from 3.695 to 2.464 and the p values from 0.0002 to 0.0142. Again the ESR1 and SLC6A4 genes were the most powerful predictors for AD and the ACE and ACP1A the less powerful predictors. These results indicate that the use of the four additional non-APOE genes provide a significant improvement over the prediction of the presence of AD (r²⁼078, p<0.0001) compared to APOE alone (r²=0.162, p<0.0001) and that all five genes together produces a significantly higher r² (0.24) than for the APOE gene alone (Chi square=7.7, d. f=1, one-sided p=0.0027).

TABLE 4
Individual AD Genes

| | | | | ANOVA | | | | | | regression analysis | | |
|---|---|---|---|---|---|---|---|---|---|---|---|---|
| Gene | SNP | N | Genotypes | AD Score | S.D. F-ratio | | p | F_{L}-ratio | P_{L} | r | r² | p |
| ACPI | Taq1 | 204 | 11 | 1.304 | .46 | | | | | | | |
| | | 189 | 12 | 1.381 | .49 | | | | | | | |
| | | 40 | 22 | 1.500* | .50 | 3.30 | .0378 | 6.44 | .011 | .121 | .014 | .011 |
| APOE | e2-4 | 248 | non4/non4 | 1.218 | .41 | | | | | | | |
| | | 134 | het | 1.560* | .50 | | | | | | | |
| | | 26 | 4/4 | 1.808*# | .40 | 39.70 | <.0001 | 78.79 | <.00001 | .403 | .1626 | <.0001 |
| ESRI | Xba 1 | 133 | 11 | 1.458* | .50 | | | | | | | |
| | | 220 | 12 | 1.264 | .44 | | | | | | | |
| | | 77 | 22 | 1.415* | .49 | 8.00 | .0004 | 1.94 | .164 | .188 | .0352 | .0001 |
| SLC6A4 | sertrans | 69 | non12 | 1.217 | .41 | | | | | | | |
| | | 179 | het | 1.346 | .48 | | | | | | | |
| | | 174 | 12/12 | 1.402* | .49 | 3.76 | .024 | 6.97 | .0086 | .128 | .0163 | .0086 |
| ACE | C/T | 100 | 11 | 1.450 | .50 | | | | | | | |
| | 22251 | 227 | 12 | 1.304 | .46 | | | | | | | |
| | | 99 | 22 | 1.374 | .49 | 3.37 | .035 | 1.29 | .255 | .125 | .0156 | .0097 |

*significantly different from lowest value by post hoc Tukey test.
#significantly different from second lowest value by post hoc Tukey test

TABLE 5
Multivariate Regression Analysis of Five Genes
versus the AD Score.

| Gene | B | S.E. | Beta | T | p |
|---|---|---|---|---|---|
| APOE | .063245 | .007091 | .396734 | 8.919 | .0000 |
| SLC6A4 | .109987 | .029829 | .163309 | 3.687 | .0003 |
| ESR1 | .075667 | .021335 | .157264 | 3.547 | .0004 |
| ACP1A | .082213 | .033017 | .110476 | 2.490 | .0132 |
| ACE | .054539 | .025838 | .093571 | 2.111 | .0354 |
| (Constant) | .920997 | .056538 | | 16.290 | .0000 |
| r | .49256 | | | | |
| r2 | .24262 | | | | |
| Adjusted r² | .23283 | | | | |
| F = | 24.79437 | | | | |
| p | <.00001 | | | | |

TABLE 6
Multivariate Regression Analysis of the Four Non-APOE
Genes versus the AD score.

| Gene | B | .E. | Beta | T | p |
|---|---|---|---|---|---|
| ESR1 | .083744 | .022664 | .175140 | 3.695 | .0002 |
| SLC6A4 | .089233 | .031553 | .134039 | 2.828 | .0049 |
| ACE | .068638 | .027447 | .118440 | 2.501 | .0128 |
| ACP1A | .085947 | .034887 | .116783 | 2.464 | .0142 |
| (Constant) | 1.058841 | .057911 | | 18.284 | .0000 |
| r | .27982 | | | | |
| r² | .07830 | | | | |
| Adjusted r² | .06933 | | | | |
| F = | 8.72866 | | | | |
| p | <.00001 | | | | |

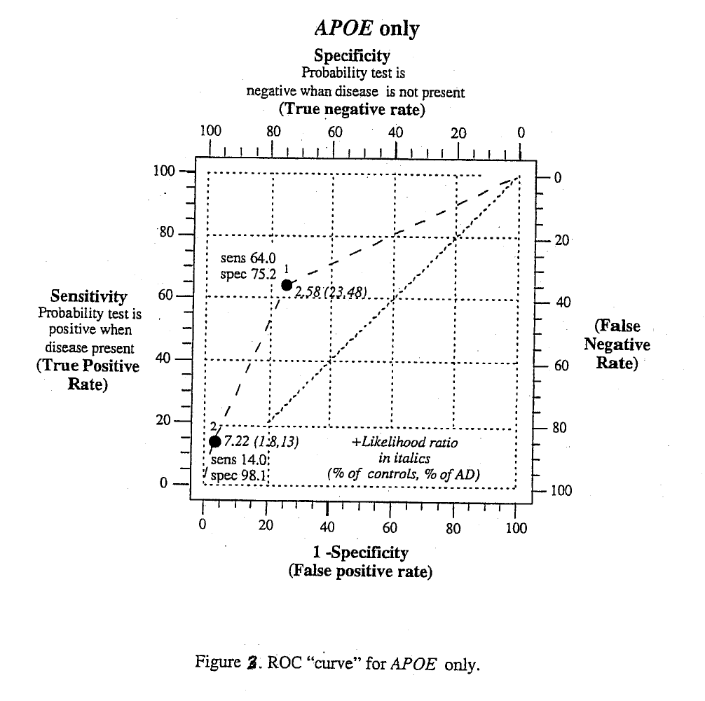

ROC plots. One of the most powerful methods of assessing the effectiveness of a set of tests in predicting the presence or absence of a given disease state is the use of Receiver Operating Characteristic (ROC) plot (Metz et al.; Zweig et al.; and Hanley et al.). The ROC plot depicts the overlap between the two distributions by plotting the sensitivity versus 1-specificity for the complete range of decision thresholds. On the y-axis is sensitivity, or the true-positive fraction [defined as (number of true-positive test results)/(number of true-positive+number of false-positive test results)]. This has also been referred to as the positivity in the presence of a disease based on calculations for the affected group. On the x-axis is the false-positive fraction, or 1-specificity [defined as (number of false-positive results)/(number of true-negative+number of false-positive results)]. This is an index of specificity and is calculated from the unaffected group (Zweig et al.). In summary, sensitivity=true-positive results/total patients with the disease and specificity=true-negative results/total patients without the disease (Griner et al.). This allows the determination of the+likelihood ratio for the presence of disease for each of the sensitivity-specificity pairs. Finally, the program calculates the area under the curve, a further measure of the effectiveness of the test (Hanley et al.). FIG. 3 shows a ROC plot using only the APOE genotypes. In this case, rather than being a curve, since there are only two values, e4/e4 and e4/non4, it really consists of two dots, 1 and 2. For e4 heterozygotes the sensitivity was 64.0 and the specificity was 75.2 with a + likelihood ratio of 2.5 over those with a 0 score. For e4/e4 homozygotes, the sensitivity was low, 14, while the specificity was high, 98.1. Here the positive likelihood ratio was 7.22. This diagram illustrates many of the problems with using APOE alone to predict AD. While the specificity of e4/e4 homozygosity is quite high (98.1%), because the frequency of this genotype is low in both the general population (1.8%) and in the population of AD subjects (13%), the population wide sensitivity is low (14%). In addition, while the frequency of e4 heterozygosity in the general population (23%) and in the population of AD subjects (48%) is higher, and thus the sensitivity is higher (64%), the specificity is lower (75.2%).

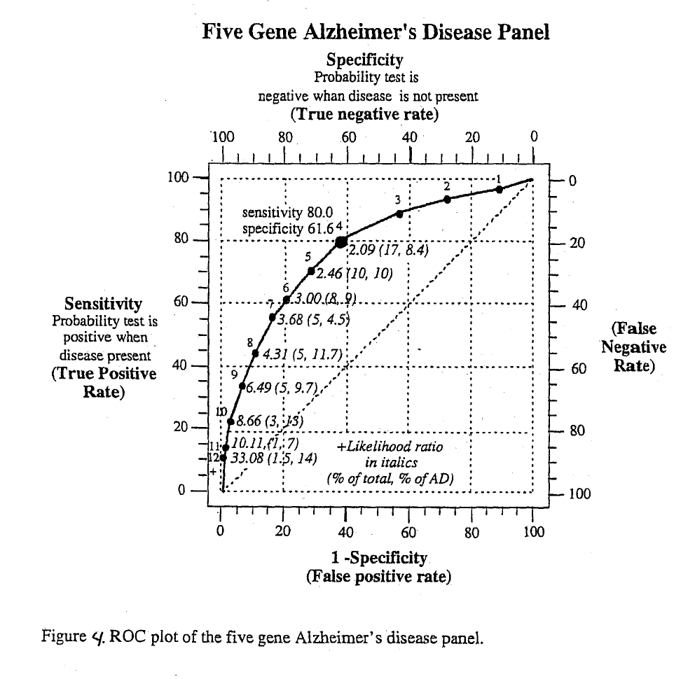

To determine if the addition of four genes discussed above could provide a significant improvement over the use of the APOE gene alone, we calculated an additive AD risk score. For each gene the genotypes most associated with AD were scored as 2, those least associated were scored 0, and the intermediate genotype was scored, 0, 1 or 2 depending upon whether it was truly intermediate, closer to the 0 genotype or closer to the 2 genotype. Because the F-ratios were five fold higher than for these genes, the APOE e4/e4 genotype was scored 10, the e4/non-e4 was scored 5, and the non-e4/non-e4 genotype was scored 0. For each individual a total AD risk score was computed by adding together the five gene scores. The results in terms of this ROC plot are shown in FIG. 4. In contrast to the use of the APOE gene alone, there are now 12 different scores and thus 12 different points on the curve. The point for an AD score of 4 represented the maximum for sensitivity (80%) for specificity (61.6%). Most importantly, it can be seen that the positive likelihood ratios are considerably higher than for APOE alone. Thus, including only those with a likelihood ratio of greater than 2.58 for the APOE e4 heterozygotes, the positive likelihood ratios ranged from 33.08 for a score of 12 or greater to 3.0 for those with a score of 6 or greater. As described above, only 1.8% of the general population were e4/e4 homozygotes, with a positive likelihood ratio of 7.22 and 23% of the population were e4 heterozygotes with a positive likelihood ratio of 2.58. Summing these gives and average of likelihood ratio of 2.9 for 24.8% of the population. By contrast, with the five gene panel, there was an average likelihood ratio of 6.38 for 28.5% of the general population, a 2.5 fold improvement.

In conclusion, the addition of four more AD-risk associated genes, SLC6A4, ESR1, ACP 1, and ACE has significantly enhanced the usefulness of genetic testing to determine an individuals risk for AD. This significant enhancement can be demonstrated in the following ways: by ANOVA, each of the genes individually was significantly associated with AD, with standard p values ranging from 0.038 to <0.00012 (Table 4); by multivariate regression analysis, in the presence of the APOE gene, the p values for the individual gene ranges from 0.035 to 0.0003; all five genes accounted for 24% of the variance of AD (p<0.0001). Of this the four non-APOE genes accounted for 7.8% of the variance, p<0.0001, and the p value for the individual genes ranged from 0.014 to 0.0002; and, analysis by ROC plot showed that while APOE gene had only two points with a positive likelihood ratio of 2.58 for e4 heterozygotes (23% of the population) and 7.22 for the rare e4/e4 homozygotes (1.89% of the population), for the five panel test there were 12 points and the positive likelihood ratios for the scores of 6 to 12 were 3.0, 3.68, 4.3,8.66, 10.11 and 33.08 respectively.

This example demonstrates that by using the additive effect of five genes, APOE, SLC6A4, ESR1, ACP 1, and ACE the ability to predict AD risk is significantly enhanced over the use of APOE alone. The unique aspect is the use of these five genes in an additive fashion and the use of ROC plots to evaluate the risk of a given individual tested for these five genes to develop AD. This approach is not polymorphism specific. A number of different polymorphisms at the SLC6A4, ESR1, ACP 1, and ACE gene could be equally useful and this patent covers the concept for any combination of polymorphisms at these genes and their additive effect. Furthermore, it will be readily apparent to the skilled artisan that additional genes may supplement or replace one or more of the five genes, as long as the new genes contribute a greater relative risk score when compared to APOE clone. Similar expansion of risk associated alleles and genes are contemplated for analysis of LOAD-risk associated genes as well as the identification of genes associated with other polygenic traits, disorders or diseases.

### Discussion

APOE only. The association of the APOE gene with LOAD has been replicated in many studies (Farrer et al. 1997). Thus, it would be expected that if one examined the additive effect of five different genes, the r² for APOE might be higher than for all the other four genes combined. This was the case. Thus, when all five genes were examined together in a multivariate logistic regression analysis (Table 3A), for both sets together the APOE gene accounted for 70% (10 times the weight) of the total r². Despite this, as discussed below, the other genes made a significant contribution to estimating the risk of LOAD. Similar results were seen for AD, but with a different combination of candidate genes and alleles

Other genes significant in both sets for LOAD. In addition to APOE, when examined individually the ACE and the ESR1 Xba I genes were significant in both set 1 and set 2. There is no strong interaction effect between these two genes. However their additive effect decreased from 0.065 to 0.039, when APOE was added.

Other genes significant in one set but not the other for LOAD. When the ESR1 gene was examined using the Pvu II polymorphism, the results were significant by chi square analysis for set 1 (p≦0.0074) and set 1 and 2 combined (p≦0.0037), but not for set 2 (p≦0.274). The results were the same, but with lower p values, by r² testing. Using chi square analysis the G->A-387 polymorphism and r² analysis, the PNMT gene was significant for set 2 but not set 1. Using r² analysis the SLC6A4 gene was significant for set 2 and the total of both sets, but not for set 1.

Other genes not significant in either set for LOAD. The ACP1 gene was not significant in either set.

The bootstrap simulation provided similar ODD ratio estimates for different subsets consistent with the probability that the variation from set 1 to set 2 was due to the characteristics of polygenic disorders, such as genetic heterogeneity. These results confirm that some genes are significant in both sets, some are be significant in only one set, and some are be significant in neither set. With some genes being significant in one set but not replicated in a second set, and visa versa, and some genes being significant only when a larger number of cases were involved, these results mimic the field of studies of single genes in polygenic disorders.

In set 2 of the LOAD examples, where the relative contribution of the APOE gene was much less than it was in set 2, the role of the non-APOE genes was even greater (see Table 3A). These observations illustrate the concept of genetic heterogeneity in polygenic disorders and support the concept presented here that in different sets of patients, different combinations of candidate genes can contribute to the same phenotype, but that the sum of the r² value for all of the candidate genes may be similar. Here it was 0.24 for set 1 and 0.21 for set 2.

ROC Plots. One result of this work was to determine if the additive effect of several other candidate genes might improve upon the estimates of the risk for LOAD and the area under the ROC curves seen with APOE only. When all non-APOE genes were examined the area under the curves were less than for the APOE gene alone (FIG. 1B). This was expected since the APOE gene accounted for more than half of the variance of all five genes. However, the area under the curve of the other genes was similar (0.694 versus 0.66) despite the fact that there were considerable differences in the p values of these other genes between set 1 and set 2. This is consistent with the a priori hypothesis that examining the additive effect of multiple genes provides a more reproducible result in polygenic disorders than examining genes one at a time. When the APOE gene was included (FIG. 1C) the area under the ROC plot increased to 0.75 for set 1 and set 2. This was an improvement over the results for the APOE gene alone. It was clear however, that the method of gene scoring was under estimating the relative contribution of the APOE gene since all genes were scored 0, 1 or 2 despite their relative importance. The gene score for APOE and other genes required weighting to compensate for their greater or lesser relative contribution. Now the area under the ROC plot curves were the highest for all the scores we examined: 0.79 for set 1 and 0.75 for set 2, indicating that the use of weighted gene scores is the optimal technique. The two curves were similar indicating that when multiple genes are used, even when the genes involved and the relative association of the genes with the phenotype vary considerably from set to set, the final results can be similar. The use of additional genes and weighted scores also increased the range of the relative risk figures (see underlined figures in FIG. 1). They now ranged from 0.15 to 11.1 for both weighted sets combined compared to 0.58 to 4.08 for APOE alone. Thus, for the purposes of predicting a given individual's relative risk for LOAD, this five-gene test is potentially more clinically useful than the use of APOE alone.

A plot of the separation of each individual weighted PG score for controls versus LOAD cases for sets 2 is shown in FIG. 2, with the weighted PG score on the ordinate. At a break point of >11, 69.6% of the LOAD cases had a score of greater than 11 and 70.3% of the controls had a score of 11 or less. This is the point at which the sensitivity and specificity are maximized. As shown in FIG. 1D, a considerable number of individuals at risk for LOAD are likely to receive a score with a specificity of 90% or greater. As shown in FIG. 2, there were three control subjects with a weighted score of equal to or greater than 20, compared to 16 LOAD subjects. Since the mean age of the controls was not as high as the mean age of death of the LOAD cases, these three controls could represent individuals who will develop LOAD as they get older.

An additional advantage of using a weighted gene score is that it is not necessary to be overly concerned about which of the candidate gene should be included in an eventual test for the phenotype in question since they could all be used. Any gene with small contribution would have a small r² and a small weight and would be essentially factored out. However, to decrease the cost of the test it is reasonable to exclude those genes with low r² value.

Multiple SNPs per gene. If two SNPs are in total linkage disequilibirum (LD), one SNP can be discarded. If two SNPs are in total equilibrium, they can be examined separately. If two (or more) SNPs are in partial LD, as was the case here for both the PNMT and the ESR1 genes, the usual approach is to attempt to examine haplotypes. However, this is not always easy. In the absence of family data, statistically probable haplotypes have to be used. In the present examples, there is a priori evidence for both of the genes with two SNPs that the greatest association might be with double heterozygotes (Mann et al. 2001; Ushiyama et al. 1998). When this was examined for the ESR1 gene for both sets combined, the p value for the nine way ANOVA was higher (p≦0.0002) than the p value for the chi square analysis for the Xba I alone (p≦0.00005). For the PNMT gene, the r² for the combined polymorphisms for both sets (0.0055) was less than for PNMT G->A-387 alone (0.0071). Thus, these results demonstrate that the present technique of examining the additive effect of multiple genes, including multiple SNPs of a single gene independently is easier and may provide more power than using haplotypes.

### Example III

Multi Gene Predictive Test for a Polygenic Disorder.

The present invention provides new techniques which incorporate the unique characteristics of polygenic disorders. In a preferred embodiment, the technique comprises the following approach:

1. Accumulate a set of candidate genes and their respective polymorphisms that have been shown to be associated with the phenotype in question in at least one, and preferably several, but not necessarily all studies;

2. Genotype a number of subjects expressing the trait, disease or disorder and a number of controls not expressing the trait, disease or disorder at each of these genes;

3. Determine the score (in a preferred embodiment 0, 1 or 2) for each genotype;

4. determining a combined relative risk score based on adding together relative risk scores for the genes determined by the logistic regression analysis to continue to contribute to the polygenic trait, disease or disorder; and

5. examining the combined relative risk score in a Receiver Operator Characteristic (ROC) plot, wherein the ROC curves plot the different values of the test against the specificity and 1-sensitivity of each value and wherein if a combined relative risk score is greater than the risk score associated with an individual allele for two or more of said genes, the detection of said two or more genes represent and improved test for predicting the presence or predisposition to said trait, disease or disorder when compared to a method which detects only one of said alleles of said genes.

Replications of the methods of identifying genes contributing to polygenic traits can fine tune the test. If the area under the ROC curve is less than 0.75, it is likely that additional genes will need to be identified. If the area under the curve is 0.75 or greater it should now be possible to perform a multigene test on any individual such that they can be assessed for their risk of developing the disorder in question. For a given individual, once the genotyping is completed the weighted PG score can be calculated, and the specificity and sensitivity of that score read off the ROC plot, and the relative risk for the disorder determined. The use of a multi-gene score, and the ability of this technique to work even in the presence of considerable genetic heterogeneity and a small effect size of each gene, compensates for the unique aspects of polygenic disorders. In relation to AD and/or LOAD, either the present sets, or expanded sets of genes will provide a test for identifying given individuals at risk for Alzheimer's Disease and/or Late-Onset Alzheimer's Disease.

### BIBLIOGRAPHY

ACMG/ASHG (1995) Statement on use of apolipoprotein E testing for Alzheimer Disease. JAMA 274:1627-1629.

Alvarez, R., Alvarez, V., Lahoz, C. H., Martinez, C., Pena, J., Sanchez, J. M., Guisasola, L. M., Salas-Puig, J., Moris, G., Vidal, J. A., Ribacoba, R., Menes, B. B., Uria, D., Coto, E. (1999). Angiotensin converting enzyme and endothelial nitric oxide synthase DNA polymorphisms and late onset Alzheimer's disease. J Neurol Neurosurg Psychiatry 67: 733-6. 99.

Appel E, Eisenberg S, Roitelman J (1995) Improved PCR amplification/Hhal restriction for unambiguous determination of apolipoprotein E alleles. Clin Chem 41:187-90.

Axelrod J, Weinshilboum R (1972) Catecholamines. New Eng.J.Med. 287:237-242.

Battersby S, Ogilvie A D, Smith C A D, Blackwood D H R, Muir W J, Quinn J P, Flnk G, Goodwin G M, Harmar A J (1996) Structure of a variable number tandem repeat of the serotonin transporter gene and association with affective disorder. Psychiat.Genet. 6:177-181.

Bottini N, MacMurray J, Peters W, Rhostamkhani M, Comings D E (2002a) Association of the acid phosphatase (ACP1) gene with triglyceride levels in obese post-menopausal women: potential relevance to the development of metabolic syndrome. (submitted).

Bottini N, MacMurray J, Touretellotte W W, CUratalo P, Rhostamkani M, Comings D E (2002b) The ACP1 locus: A possible prognostic factor in multiple sclerosis. (Submitted)

Bottini N, MacMurray J, Comings D E. (2002c). Role of the ACP1 gene in Tourette Syndrome. (submitted).

Brandi, M. L., Becherini, L., Gennari, L., Racchi, M., Bianchetti, A., Nacmias, B., Sorbi, S., Mecocci, P., Senin, U., Govoni, S. Association of the estrogen receptor alpha gene polymorphisms with sporadic Alzheimer's disease. Biochem Biophys Res Commun 265: 335-8. 99.

Burke W J, Chung H D, Nakra B R, Grossb erg G T, Joh T H (1987) Phenylethanolamine N-methyltransferase activity is decreased in Alzheimer's disease brains. Ann. Neurol. 22:278-280.

Comings D E (1998) Why Different Rules are Required for Polygenic Inheritance: Lessons from Studies of the DRD2 Gene. Alcohol 16:61-70.

Comings D E (2002) The Real Problem with Association Studies. Am. J. Med. Genet. (Neuropsychiatric Genetics) (in press).

Comings D E, Comings B G, Muhleman D, Dietz G, Shahbahrami B, Tast D, Knell E, Kocsis P, Baumgarten R, Kovacs B W, Levy D L, Smith M, Kane J M, Lieberman J A, Klein D N, MacMurray J, Tosk J, Sverd J, Gysin R, Flanagan S (1991) The dopamine D2 receptor locus as a modifying gene in neuropsychiatric disorders. J.Am.Med.Assn. 266:1793-1800.

Comings D E, Gade-Andavolu R, Gonzalez N, Wu S, Lesieur H R, Rugle L J, Rosenthal R J (2001) The additive effect of neurotransmitter genes in pathological gambling. The interaction of 31 candidate genes. Clinical Genetics 60:107-116.

Comings D E, Gade-Andavolu R, Gonzalez N. Wu S, Muhleman D, Blake H, Chiu F, Wang E, Farwell K, Darakjy S, Baker R, Dietz G, Saucier G, MacMurray J P (2000a) Multivariate analysis of associations of 42 genes in ADHD, ODD and conduct disorder. Clin Genet 58: 31-40.

Comings D E, Gade-Andavolu R, Gonzalez N, Wu S, Muhleman D, Blake H, Dietz G, Saucier G, MacMurray J P (2000b) Comparison of the role of dopamine, serotonin, and noradrenaline genes in ADHD, ODD and conduct disorder: multivariate regression analysis of 20 genes. Clin Genet 57:178-96.

Comings D E, Gade-Andavolu R, Gonzalez N, Wu S, Muhleman D, Blake H, Mann M B, Dietz G, Saucier G, MacMurray J P (2000c) A multivariate analysis of 59 candidate genes in personality traits: the temperament and character inventory. Clin Genet 58:375-85.

Comings D E, Gade R, Wu S, Chiu C, Dietz G, Muhleman D, Saucier G, Ferry L, Burchete R, Johnson P, Verde R, MacMurray J P (1997) Studies of the potential role of the dopamine D1 receptor gene in addictive behaviors. Molecular Psychiatry 2:44-56.

Comings D E, Gonzalez N, Wu S, Saucier G, Johnson P, Verde R, MacMurray J P (1999) Association of the dopamine DRD3 receptor gene with cocaine dependence. Molecular Psychiatry 4:484-487.

Comings D E, MacMurray J M (2000) Molecular Heterosis: A Review. Molecular Genetics and Metabolism 71:19-31.

Comings D E, Rostamkhani M, MacMurray J, lacono W G, McGue M (2002a) Association between the acid phosphastase (ACP1) gene and teenage smoking. J.Am.Acad.Child & Adolescent Psychiatry (in press).

Comings D E, Rostamkhani M, McGue M, Bottini N, MacMurray J (2002b) Association Between Depression and the Low Molecular Weight Cytosolic Acid Phosphatase Gene ACP1*A. Neuroscience Letters (in press).

Comings D E, Wu H, Chin C, Ring R H, Dietz G, Muhleman D (1996) Polygenic inheritance of Tourette syndrome, stuttering, ADHD, conduct and oppositional defiant disorder: The Additive and Subtractive Effect of the three dopaminergic genes-DRD2, DβH and DAT1. Am.J.Med.Gen. (Neuropsych. Genet.) 67:264-288.

Daly G, Hawi Z, Fiztgerald M, Gill M (1999) Mapping susceptability loci in attention deficit hyperactivity disorder: Preferential transmission of parental alleles at DAT1, DBH and DRD5 to affected children. Molecular Psychiatry 4:192-196.

Devlin, B., Roeder, K. Genomic control for association studies. Biometrics 55(4), 997-1004. 99.

Devlin B, Roeder K, Bacanu S-A (2001) Unbiased methods for population-based association studies. Genetic Epidemiology 21:273-284.

Dissing J, Johnsen A H (1992) Human red cell acid phosphatase (ACP1): the primary structure of the two pairs of isozymes encoded by the ACP1*A and ACP1*C alleles. Biochim Biophys Acta 1121:261-8.

Ebstein R F, Kotler M, Cohen H, Segman R, Gritsenko I, Nemanov I, Lerer B, Kramer I, Zer-Zion M, Kletz I (1996) Excess dopamine D4 receptor exon III (DRD4) seven repeat allele in opioid dependent subjects. Am.J.Hum.Genet. 59:A92.

Ernst M, Zametkin A J, Matochik J A, Jons P H, Cohen R M (1998) DOPA decarboxylase activity in attention deficit hyperactiviy disorder adults. A [fluorine-18] fluorodopa position emission tomographic study. J.Neuroscience 18:5901-5907.

Faraone S V, Biederman J, Weiffenbach B, Keith T, Chu M P, Weaver/A., Spencer T J W T E, Frazier J, Cleves M, Sakal J (1999) Dopamine D4 gene 7-repeat alleles and attention deficit hyperactivity disorder. American Journal of Psychiatry 156:768-770.

Farrer, L. A., Cupples, L. A., Haines, J. L., Hyman, B., Kukull, W. A., Mayeux, R., Myers, R. H., Pericak-Vance, M. A., Risch, N., van Duijn, C. M. Effects of age, sex, and ethnicity on the association between apolipoprotein E genotype and Alzheimer disease. A meta-analysis. APOE and Alzheimer Disease Meta Analysis Consortium. JAMA 278; 1349-56. 1997.

Farrer, L. A., Sherbatich, T., Keryanov, S. A., Korovaitseva, G. I., Rogaeva, E. A., Petruk, S., Premkumar, S., Moliaka, Y., Song, Y. Q., Pei, Y., Sato, C., Selezneva, N. D., Voskresenskaya, S., Golimbet, V., Sorbi, S., Duara, R., Gavrilova, S., St George-Hyslop, P. H., Rogaev, E. I. Association between angiotensin-converting enzyme and Alzheimer disease. Arch Neurol 57: 210-4. 2000.

Gill M, Daly G, Heron S, Hawi Z, Fitzgerald M Confirmation of association between attention deficit disorder and a dopamine transporter polymorphism. Molecular Psychiatry 2:311-313. 1997.

Griner P F, Mayewsi R J, Mushlin A I, Greenland P (1981) Selection and interpretation of diagnostic tests and procedures. Annals Int. Med. 94:555-600. 1981.

Hamer D, Sirota I (2000) Beware the chopsticks gene. Molecular Psychiatry 5:11-13.

Hanley J A, McNeil B J The meaning and use of the area under a receiver operating characteristic (ROC) curve. Radiology 143:29-36. 1982.

Hirschhorn, J. N., Lohmueller, K., Byrne, E., Hirschhorn, K. A comprehensive review of genetic association studies. Genet Med 4: 45-61. 2002.

Hixson J E, Vernier D T Restriction isotyping of human APOE by gene amplification and cleavage with Hha I. J.Lipid Res. 31:545-548. 1990.

Hu, J., Miyatake, F., Aizu, Y., Nakagawa, H., Nakamura, S., Tamaoka, A., Takahash, R., Urakami, K., Shoji, M. Angiotensin-converting enzyme genotype is associated with Alzheimer disease in the Japanese population. Neurosci Lett 277: 65-7. 99. 1999.

Ioannidis, J. P., Ntzani, E. E., Trikalinos, T. A., Contopoulos-loannidis, D. G. Replication validity of genetic association studies. Nat Genet 29: 306-9. 2001.

Isbir, T., Agachan, B., Yilmaz, H., Aydin, M., Kara, I., Eker, D., Eker, E. Interaction between apolipoprotein-E and angiotensin-converting enzyme genotype in Alzheimer's disease. Am J Alzheimers Disease and Other Demenias 16: 205-10. 2001.

Ji, Y., Urakami, K., Wada-Isoe, K., Adachi, Y., Nakashima, K. Estrogen receptor gene polymorphisms in patients with Alzheimer's disease, vascular dementia and alcohol-associated dementia. Dement Geriatr Cogn Disord 11: 119-22. 2000.

Kaiser, R., Muller-Oerlinghausen, B., Filler, D., Tremblay, P. B., Berghofer, A., Roots, I., Brockmoller, J. Correlation between serotonin uptake in human blood platelets with the 44-bp polymorphism and the 17-bp variable number of tandem repeat of the serotonin transporter. Am J Med Genet 114: 323-8. 2002.

Kobayashi S, Inoue S, Hosoi T, Ouchi Y, Shiraki M, Orimo H (1996) Association of bone mineral density with polymorphism of estrogen receptor gene. J.Bone Miner.Res. 11:306-311. 1996.

Kunugi, H., Ueki, A., Otsuka, M., Isse, K., Hirasawa, H., Kato, N., Nabika, T., Kobayashi, S., Nanko, S. Alzheimer's disease and 5-HTTLPR polymorphism of the serotonin transporter gene: no evidence for an association. Am J Med Genet 96: 307-9. 2000.

Lahoste G J, Swanson J M, Wigal S B, Glabe C, Wigal T, King N, Kennedy J L Dopamine D4 receptor gene polymorphism is associated with attention deficit hyperactivity disorder. Molecular Psychiatry 1:121-124. 1996.

Lambert, J. C., Harris, J. M., Mann, D., Lemmon, H., Coates, J., Cumming, A., St-Clair, D., Lendon, C. Are the estrogen receptors involved in Alzheimer's disease? Neurosci Lett 306: 193-7. 2001.

Li, T., Holmes, C., Sham, P. C., Vallada, H., Birkett, J., Kirov, G., Lesch, K. P., Powell, J., Lovestone, S., Collier, D. Allelic functional variation of serotonin transporter expression is a susceptibility factor for late onset Alzheimer's disease. Neuroreport 8: 683-6. 97. 1997.

Mann M B, Wu S, Rostamkhani M, Tourtellotte W, MacMurray J P, Comings D E Phenylethanolamine N-methyltransferase (PNMT) gene and early-onset Alzheimer's disease. Am.J. Medical Genetics (Neuropsychoatric Genetics) 105:312-316. 2001.

Maruyama, H., Toji, H., Harrington, C. R., Sasaki, K., Izumi, Y., Ohnuma, T., Arai, H., Yasuda, M., Tanaka, C., Emson, P. C., Nakamura, S., Kawakami, H. Lack of an association of estrogen receptor alpha gene polymorphisms and transcriptional activity with Alzheimer disease. Arch Neurol 57: 236-40. 2000.

Mattila, K. M., Axelman, K., Rinne, J. O., Blomberg, M., Lehtimaki, T., Laippala, P., Roytta, M., Viitanen, M., Wahlund, L., Winblad, B., Lannfelt, L. Interaction between estrogen receptor 1 and the epsilon4 allele of apolipoprotein E increases the risk of familial Alzheimer's disease in women. Neurosci Lett 282: 45-8. 2000.

Metz C E Basic principles of ROC analysis. Seminars in Nuclear Medicine 8:283-298. 1978.

Morton, N. E., Collins, A. Tests and estimates of allelic association in complex inheritance. Proc Natl Acad Sci USA 95; 11389-93. 1998.

Mossner, R., Schmitt, A., Syagailo, Y., Gerlach, M., Riederer, P., Lesch, K. P. The serotonin transporter in Alzheimer's and Parkinson's disease. J Neural Transm Suppl: 345-50. 2000.

Narain, Y., Yip, A., Murphy, T., Brayne, C., Easton, D., Evans, J. G., Xuereb, J., Cairns, N., Esiri, M. M., Furlong, R. A., Rubinsztein, D. C. The ACE gene and Alzheimer's disease susceptibility. J Med Genet 37: 695-7. 2000.

Ogilvie A D, Battersby S, Bubb V J, Flnk G, Hamaar A J, Goodwin G M, Smith C A D Polymorphism in the serotonin transporter gene associated with susceptability to major depression. Lancet 347:731-733. 1996.

Oliveira, J. R., Gallindo, R. M., Maia, L. G., Brito-Marques, P. R., Otto, P. A., Passos-Bueno, M. R., Morais, M. A. Jr, Zatz, M. The short variant of the polymorphism within the promoter region of the serotonin transporter gene is a risk factor for late onset Alzheimer's disease. Mol Psychiatry 3: 438-41. 1998.

Oliveira J R, Shimokomaki C M, Brito-Marques P R, Gallindo R M, Okuma M, Maia L G, Otto P A, Passos-Bueno M R, Zatz M The association of the short variant of the 5-HTTPLR polymorphism and the apoE epsilon4 allele does not increase the risk for late onset Alzheimer's disease. Mol Psychiatry 4:19-20. 1999.

Paganini-Hill A, Henderson V W Estrogen replacement therapy and risk of Alzheimer disease. Arch. Int. Med. 156:2213-2217. 1996.

Reich D E and Goldstein D B Detecting association in a case-control study while correcting for population stratification. Genet Epidemiol 20:: 4-16. 2001.

Rieder, M. J., Taylor, S. L., Clark, A. G., Nickerson, D. A. Sequence variation in the human angiotensin converting enzyme. Nat Genet 22: 59-62. 1999.

Risch, N. J. Searching for genetic determinants in the new millennium. Nature 405: 847-56. 2000.

Rowe D C, Stever C, Giedinghagen L N, Gard J M C, Cleveland H H, Terris S T, Mohr J H, Herman S, Abramowitz A, Waldman I D Dopamine DRD4 receptor polymorphism and attention deficit hyperactivity disorder. Molecular Psychiatry 3:419-426. 1998.

Russlee V, de Villiers A, Sagvolden T, Lamm M, Taljaard J Differences between electrically-ritalin- and D-amphetamine-stimulated release of [³H]dopamine from brain slices suggest impaired vesicular storage of dopamine in an animal model of Attention-Deficit Hyperactity Disorder. Behav.Brain Res. 94:163-171. 1998.

Savaskan, E., Hock, C., Olivieri, G., Bruttel, S., Rosenberg, C., Hulette, C., Muller-Spahn, F. Cortical alterations of angiotensin converting enzyme, angiotensin II and ATI receptor in Alzheimer's dementia. Neurobiol Aging 22: 541-6. 2001.

Sensabaugh G F, Lazaruk K A A TaqI site identifies the *A allele at the ACP1 locus. Hum Mol Genet 2:1079. 1993.

Shimohama S, Fujimoto S, Chachin M, Taniguchi T, Perry G, Whitehouse P J, Kimura J Alterations of low molecular weight acid phosphatase protein level in Alzheimer's disease. Brain Res 699:125-9. 1995.

Shimohama S, Fujimoto S, Taniguchi T, Kameyama M, Kimura J Reduction of low-molecular-weight acid phosphatase activity in Alzheimer brains. Ann Neurol 33:616-21. 1993.

Spencer N, Hopkinson D A, Harris H Quantitative differences and gene dosage in the human red cell acid phosphatase polymorphism. Nature 201:299-300. 1964.

Spielman R S, Ewens W J The TDT and other family-based tests for linkage disequilibrium and association. Am.J.Hum.Genet. 59:983-989. 1996.

Tang M-X, Jacobs D, Stern Y, Marder K, Schofield P, Gurland B, Andrews H, Mayeux R (1996) Effect of oestrogen during menopause on risk and age at onset of Alzheimer's disease. Lancet 348:429-432.

Tanino H, Yoshida J, Yamamoto R, Kobayashi Y, Shimohama S, Fujimoto S (1999) Abundance of low molecular weight phosphotyrosine protein phosphatase in the nerve-ending fraction in the brain. Biol Pharm Bull 22:794-8.

Tsai, S. J., Hong, C. J., Liu, T. Y., Cheng, C. Y., Liu, H. C. Association study for a functional serotonin transporter gene polymorphism and late-onset Alzheimer's disease for Chinese patients. Neuropsychobiology 44: 27-30. 2001.

Ushiyama T, Ueyama H, Inoue K, Nishioka J, Ohkubo J, Hukuda S (1998) Estrogen receptor gene polymorphism and generalized osteoarthritis. J. Rheumatology 25:134-137.

Waldman I D, Rowe D C, Abramowitz S, Kozel T, Mohr J H, Sherman S L, Cleveland H H, Sanders M L, Gard J M C, Stever C (1998) Association and linkage of the dopamine transporter gene and attention-deficit hyperactivity disorder in children: Heterogeneity owing to diagnostic subtype and severity. Am.J.Hum.Genet. 63:1767-1776.

Wu S, Comings D E (2000) Two single polymorphisms in the promoter region of the human phenylethanolamine N-methyltransferase PNMT gene. Psychiatric Genetics 9:187-188.

Zhang S, Zhai H (2001) Quantitative similarity-based association tests using population samples. Am.J.Hum. Genet. 69:601-604.

Zill, P., Padberg, F., de Jonge, S., Hampel, H., Burger, K., Stubner, S., Boetsch, T., Jurgen Moller, H., Ackenheil, M., Bondy, B. Serotonin transporter (5-HTT) gene polymorphism in psychogeriatric patients. Neurosci Lett 284:113-5. 2000.

Zuliani, G., Ble', A., Zanca, R., Munari, M. R., Zurlo, A., Vavalle, C., Atti, A. R., Fellin, R. Genetic polymorphisms in older subjects with vascular or Alzheimer's dementia. Acta Neurol Scand 103: 304-8. 2001.

Zweig M H, Campbell G (1993) Receiver-operating characteristic (ROC) plots: a fundamental evaluation tool in medicine. Clinical Chemistry 39:561-577.

1. A method of determining whether an individual is at an increased risk of developing or having Alzheimer's Disease (AD), comprising analyzing the subject's genetic material for the presence of at least one AD-risk associated allele for at least two different genes, wherein the presence of said at least one allele for at least two genes is indicative of an increased risk of having or developing AD.

2. A method as in claim 1 wherein the AD-risk associated alleles are selected from the group consisting of:
the e4 allele of the APOE gene;
the C>T 22251 polymorphism of the ACE gene;
the Xbal polymorphism of the ESR gene
the VNTR polymorphism of the SLC6A4 gene; and
the ACP1*A polymorphism of the ACP1 gene.

3. The method of claim 2, wherein the presence of an allele of all of the genes of said group is determined.

4. The method of claim 1, wherein the presence of a AD-risk associated allele of two of said genes indicates a risk for AD.

5. The method of claim 1 wherein the subject's genetic material is analyzed for the presence of the e4 allele of the APOE gene; the C>T 22251 polymorphism of the ACE gene; and the Xbal polymorphism of the ESR gene.

6. A method of determining whether an individual is at an increased risk of developing or having Late Onset Alzheimer's Disease (LOAD), comprising analyzing the subject's genetic material for the presence of at least one LOAD-risk associated allele for at least two different genes, wherein the presence of said at least one allele for at least two genes is indicative of an increased risk of having or developing LOAD.

7. A method as in claim 6 wherein the LOAD-risk associated alleles are selected from the group consisting of:
the e4 allele of the APOE gene;
the C>T 22251 polymorphism of the ACE gene;
the Xbal polymorphism of the ESR gene;
the VNTR polymorphism of the SLC6A4 gene;
the G>A-148 polymorphism of the PNMT gene; and
the G>A-353 polymorphisms of the PNMT gene; and
the PvuII polymorphism of the ESR gene.

8. The method of claim 7, wherein the presence of an allele of all of the genes of said group is determined.

9. The method of claim 7, wherein the presence of a LOAD-risk associated allele of three of said genes indicates an increased risk for LOAD.

10. The method of claim 9 wherein the LOAD-risk associated alleles are the e4 allele of the APOE gene; the C>T 22251 polymorphism of the ACE gene; and the Xbal polymorphism of the ESR gene.

11. A method of determining a treatment modality for a human subject suspected of having AD, comprising analyzing the subject's genetic material for the presence of an allele of at least two different genes selected from the group consisting of:
the e4 allele of the APOE gene;
the C>T 22251 polymorphism of the ACE gene;
the Xbal polymorphism of the ESR gene
the VNTR polymorphism of the SLC6A4 gene; and
the ACP1*A polymorphism of the ACP1 gene,
and determining a treatment on the basis of said at least two alleles.

12. A method of determining a treatment modality for a human subject suspected of having LOAD, comprising analyzing the subject's genetic material for the presence of an allele of at least two different genes selected from the group consisting of;
the e4 allele of the APOE gene;
the C>T 22251 polymorphism of the ACE gene;
the Xbal polymorphism of the ESR gene;
the VNTR polymorphism of the SLC6A4 gene;
the G>A-148 polymorphism of the PNMT gene;
the G>A-353 polymorphisms of the PNMT gene; and
the PvuII polymorphism of the ESR gene; and
determining a treatment on the basis of said at least two alleles.

13. The method of claim 12, wherein the presence of an allele of two of said genes is determined.

14. A method for developing a polygenic assay that is diagnostic for a trait, disease or disorder which comprises:
(a)identifying the trait, disease or disorder that is to be studied;
(b) creating a scale measuring the lowest and highest scores representing the phenotypic expression of the trait, disease or disorder to be studied;
(c) selecting at least two candidate genes that may contribute to said trait, disease or disorder;
(d) identifying at least one polymorphic allele associated with each of said candidate genes and the trait, disease or disorder;
(e) correlating allelic patterns of said polymorphism with said scale;
(f) comparing the association of said allelic pattern to the correlation of said candidate gene to said trait, disease or disorder;
(g) wherein the allelic patterns that are positively associated with said trait, disease or disorder are added, to form a polygenic assay that is diagnostic for the trait, disease or disorder;
wherein the assay comprises detecting the presence of the allelic patterns that are positively associated with said trait, disease or disorder.

15. A kit suitable for screening a subject to determine whether such subject is at increased risk for having or developing AD associated with the presence of a AD-risk associated gene, said kit comprising:
a) material for determining the subject's genotype with respect to at least two AD-risk associated genes;
b) suitable packaging material; and optionally
c) instructional material for use of said kit.

16. A kit as in claim 15 wherein the AD-risk associated genes are selected from the group consisting of:
the e4 allele of the APOE gene;
the C>T 22251 polymorphism of the ACE gene;
the Xbal polymorphism of the ESR gene
the VNTR polymorphism of the SLC6A4 gene; and
the ACP1*A polymorphism of the ACP1 gene.

17. A kit suitable for screening a subject to determine whether such subject is at increased risk for having or developing LOAD associated with the presence of a LOAD-risk associated gene, said kit comprising:
a) material for determining the subject's genotype with respect to at least two LOAD-risk associated genes;
b) suitable packaging material; and optionally
c) instructional material for use of said kit.

18. A kit as in claim 17 wherein the LOAD-risk associated genes are selected from the group consisting of:
the e4 allele of the APOE gene;
the C>T 22251 polymorphism of the ACE gene;
the Xbal polymorphism of the ESR gene;
the VNTR polymorphism of the SLC6A4 gene;
the G>A-148 polymorphism of the PNMT gene;
the G>A-353 polymorphisms of the PNMT gene; and
the PvuII polymorphism of the ESR gene.

19. A method for determining whether a group of genes together contribute to a polygenic trait, disease or disorder, the method comprising the steps of:
(1) genotyping two or more candidate genes having an allele significantly associated with the polygenic trait, disease or disorder;
(2) scoring one or more alleles for each gene depending upon whether the allele for the gene showed the least, intermediate, or strongest association with the polygenic trait, disease or disorder;
(3) performing multivariate logistic regression analysis to determine which of the alleles of said genes, in the presence of the other alleles of said other genes, continued to contribute to the polygenic trait, disease or disorder;
(4) determining a combined relative risk score based on adding together relative risk scores for the genes determined by the logistic regression analysis to continue to contribute to the polygenic trait, disease or disorder; and
(5) examining the combined relative risk score in a Receiver Operator Characteristic (ROC) plot, wherein the ROC curves plot the different values of the test against the specificity and 1-sensitivity of each value and wherein if a combined relative risk score is greater than the risk score associated with an individual allele for two or more of said genes, the detection of said two or more genes represent and improved test for predicting the presence or predisposition to said trait, disease or disorder when compared to a method which detects only one of said alleles of said genes.

20. The method of claim 19 which further comprises the step of (6) repeating the entire process in a second, well-matched set of subjects having the polygenic trait, disease or disorder and controls to determine the degree to which the results in the first set could be replicated in the second set.


SEQUENCE LISTING (9 sequences)

SEQ ID NO: 1 - DNA, 17 bp, Artificial Sequence
  SLC6A4 primer
ggctgygaccyrgrrtg

SEQ ID NO: 2 - DNA, 21 bp, Artificial Sequence
  SLC6A4 primer
gtcagtatcacaggctgcgag

SEQ ID NO: 3 - DNA, 22 bp, Artificial Sequence
  SLC6A4 primer
tgttcctagtcttacgccagtg

SEQ ID NO: 4 - DNA, 21 bp, Artificial Sequence
  ACP1 primer
ttcagaagaccctagcagatg

SEQ ID NO: 5 - DNA, 26 bp, Artificial Sequence
  ACP1 primer
acataatagggatcttcgataataag

SEQ ID NO: 6 - DNA, 19 bp, Artificial Sequence
  ACE primer
gcatctacacagscacggc

SEQ ID NO: 7 - DNA, 21 bp, Artificial Sequence
  ACE primer
gaactggatgatgaagctgtc

SEQ ID NO: 8 - DNA, 23 bp, Artificial Sequence
  ESR1 primer
tcagaaccattagagaccaatgc

SEQ ID NO: 9 - DNA, 20 bp, Artificial Sequence
  ESR1 primer
agggttatgtggcaatgacg